(19) United States
(12) Patent Application Publication
(10) Pub. No.: US 2026/0016393 A1
(43) Pub. Date: Jan. 15, 2026

(54) DEVICE AND METHOD FOR ANALYZING COFFEE PARTICLES

(71) Applicant: SHENZHEN DIGITIZING FLUID TECHNOLOGY CO., LTD., SHENZHEN (CN)

(72) Inventors: Chengjie WANG, SHENZHEN (CN); Yongzhi WU, SHENZHEN (CN); Ti PENG, SHENZHEN (CN)

(73) Assignee: SHENZHEN DIGITIZING FLUID TECHNOLOGY CO., LTD., SHENZHEN (CN)

(21) Appl. No.: 19/335,063

(22) Filed: Sep. 22, 2025

Related U.S. Application Data
(63) Continuation of application No. PCT/CN2023/088295, filed on Apr. 14, 2023.

(30) Foreign Application Priority Data
Mar. 22, 2023 (CN) 202310319552.8

(51) Int. Cl. G01N 15/1433; G01N 21/84; G01N 21/85; G01N 33/14

(52) U.S. Cl. CPC G01N 15/1433; G01N 21/85; G01N 33/14; G01N 2021/8466; G01N 2021/8592

(57) ABSTRACT

Provided in the present application are an analysis method and device for coffee particles. The device comprises: a vibration source configured to drive the coffee particles to vibrate at least twice; a camera configured to respectively capture images of the coffee particles after the at least two vibrations, obtaining a to-be-detected image set containing images of coffee particles with different distributions; one or more processors communicatively coupled to the camera; and a memory storing instructions executable by the one or more processors, wherein the instructions, when executed by the one or more processors, cause the one or more processors to: acquire initial recognition information of the coffee particles in each to-be-detected image of the to-be-detected image set; determine final recognition information of the coffee particles based on the initial recognition information of the coffee particles in at least some frames of to-be-detected images in the to-be-detected image set.

### CROSS-REFERENCE TO RELATED APPLICATIONS

This application is a continuation of International Patent Application No. PCT/CN2023/088295 filed on Apr. 14, 2023, entitled “Method, Apparatus, Device and Computer-Readable Storage Medium for Analyzing Coffee Particles”, which claims the priority to and benefit of Patent Application CN202310319552.8 filed on Mar. 22, 2023 and entitled “Method, Apparatus, Device and Storage Medium for Chromaticity Analysis of Coffee Particles”, both of which are hereby incorporated by reference in their entireties.

### TECHNICAL FIELD

This disclosure relates to the field of coffee measurement, and in particular to a method and a device for analyzing coffee particles.

### BACKGROUND

In coffee brewing, the grind size of coffee powder (i.e., the coarseness and uniformity of coffee powder particle sizes) determines the contact area between coffee powder and brewing water, which in turn affects the extraction rate of coffee powder. Generally speaking, the finer the coffee powder, the higher the extraction rate. Therefore, the extraction rate of coffee can be adjusted by modifying the grind size of coffee powder. Particle size analysis enables the acquisition of the particle size distribution of coffee powder, helping to study the coarseness and uniformity of coffee powder, determine whether the coffee powder is too coarse or too fine, and further adjust the coffee extraction rate by changing the coarseness of the coffee powder. One method for analyzing the particle size distribution of coffee powder involves acquiring the particle size of coffee powder by capturing images of the coffee powder and conducting image analysis.

The method for analyzing the particle size of coffee powder via image-based techniques still has room for improvement.

### SUMMARY

The present application provides a method and a device for analyzing coffee particles, which can improve the analysis accuracy of coffee particles.

A first aspect of the present application provides a device for analyzing coffee particle, comprising:
a vibration source configured to drive the coffee particles to vibrate at least twice;
a camera configured to respectively capture images of the coffee particles after the at least two vibrations, obtaining a to-be-detected image set containing images of coffee particles with different distributions;
one or more processors communicatively coupled to the camera; and
a memory storing instructions executable by the one or more processors, wherein the instructions, when executed by the one or more processors, cause the one or more processors to:
acquire initial recognition information of the coffee particles in each to-be-detected image of the to-be-detected image set; and
determine final recognition information of the coffee particles based on the initial recognition information of the coffee particles in at least some frames of to-be-detected images in the to-be-detected image set.

In some examples, the vibration source is configured to drive the coffee particles to vibrate in a first driving mode, resulting in coffee particles with a first distribution, and to drive the coffee particles with the first distribution to vibrate in a second driving mode, resulting in coffee particles with a second distribution; and the camera is configured to capture an image of the coffee particles with the first distribution, obtaining a first to-be-detected image, and to capture an image of the coffee particles with the second distribution, obtaining a second to-be-detected image; wherein the vibration frequency and/or vibration amplitude applied to the coffee particles in the first driving mode is higher than the vibration frequency and/or vibration amplitude applied to the coffee particles in the second driving mode.

In some examples, the vibration source is configured to, after driving the coffee particles with the first distribution to vibrate in the second driving mode, further drive the coffee particles to vibrate in the second driving mode at least once more; and the camera is configured to further capture an image of the coffee particles respectively after each vibration driven in the second driving mode, obtaining at least one frame of to-be-detected image.

In some examples, at least one of vibration frequency, vibration amplitude, vibration duration, or vibration area in the second driving mode is determined based on the initial recognition information of the coffee particles in the first to-be-detected image, wherein the initial recognition information of the coffee particles in the first to-be-detected image includes the quantity and/or area of the coffee particles in the first to-be-detected image.

In some examples, the initial recognition information of the coffee particles in the to-be-detected image includes the quantity of the coffee particles; the one or more processors are further caused to determine a change in the quantity of the coffee particles based on the quantity of the coffee particles in at least one frame of to-be-detected image prior to the first to-be-detected image, the first to-be-detected image, and the second to-be-detected image; the vibration source is further configured to stop the next driving of the coffee particles or continue the next driving of the coffee particles in the second driving mode when the quantity change is a decrease in quantity or the change value is less than a threshold; or to continue the next driving of the coffee particles in the first driving mode when the quantity change is an increase in quantity and the change value is greater than the threshold.

In some examples, the initial recognition information includes at least one of the following: at least one of particle size, quantity, area, volume, mass, and chromaticity of the coffee particles; at least one of quantity distribution, area distribution, volume distribution, mass distribution, and chromaticity distribution of the coffee particles in different particle size intervals;
at least one of quantity, area, volume, mass, and chromaticity of tiny coffee particles, where the tiny coffee particles are coffee particles with a particle size smaller than a first preset particle size, or with a particle size smaller than the first preset particle size and larger than a preset critical value;
information on the proportion of the tiny coffee particles among all coffee particles. The final recognition information includes at least one of final quantity distribution, final area distribution, final volume distribution, final mass distribution, and final chromaticity distribution of the coffee particles in different particle size intervals. The device further comprises a display configured to display at least one of the final quantity distribution, final area distribution, final volume distribution, final mass distribution, and final chromaticity distribution of the coffee particles in different particle size intervals.

In some examples, the initial recognition information of the coffee particles includes the particle size of the coffee particles. The one or more processors are further caused to: acquire a distortion function prior to determining the final recognition information of the coffee particles, where the distortion function indicates particle size compensation values at multiple pixel positions; perform distortion correction on the particle size of at least some coffee particles in the to-be-detected images of the to-be-detected image set based on the pixel positions of the coffee particles and the corresponding particle size compensation values, so as to obtain the distortion-corrected particle size of the coffee particles. The final recognition information of the coffee particles is determined based on the distortion-corrected particle size of the coffee particles in at least some frames of to-be-detected images in the to-be-detected image set.

In some examples, the initial recognition information of the coffee particles includes the particle size of the coffee particles. The one or more processors are further caused to: acquire a particle size compensation function prior to determining the final recognition information of the coffee particles, where the particle size compensation function indicates particle size compensation values under multiple brightness levels; respectively acquire the brightness of the regions where the coffee particles are located in at least some frames of to-be-detected images in the to-be-detected image set; for the at least some frames of to-be-detected images, compensate the particle size of the coffee particles in the to-be-detected images based on the brightness of the regions where the coffee particles are located in the to-be-detected images and the particle size compensation values, so as to obtain the compensated particle size of the coffee particles. The final recognition information of the coffee particles is determined based on the compensated particle size of the coffee particles in at least some frames of to-be-detected images in the to-be-detected image set. The device is configured with a calibration mode, wherein in the calibration mode, the camera is configured to capture an image of a calibration pattern with a preset area and located at a preset position within the field of view to obtain a calibration image, and the one or more processors are further caused to acquire the number of pixels corresponding to the calibration pattern, and determine a calibration size corresponding to one pixel based on the preset area and the number of pixels. The initial recognition information of the coffee particles includes the particle size of the coffee particles determined based on the calibration size and the number of pixels of the coffee particles in each to-be-detected image of the to-be-detected image set.

In some examples, the device further comprises an illumination light source and at least one spectral light source different from the illumination light source, wherein the to-be-detected image is an image captured when the coffee particles are illuminated by the illumination light source; the camera is further configured to capture at least one frame of raw image, which contains raw pixel values of images captured from the coffee particles when the coffee particles are respectively illuminated by the at least one spectral light source. The one or more processors are further caused to obtain one frame of representative chromaticity map based on the at least one frame of raw image, and to determine the overall chromaticity value of the coffee particles based on the one frame of representative chromaticity map.

In some examples, the one or more processors are further caused to: acquire at least one frame of initial raw image; determine invalid pixel values in the at least one frame of initial raw image, where the invalid pixel values refer to pixel values corresponding to objects other than the coffee particles in the initial raw image; and remove the invalid pixel values from the at least one frame of initial raw image to obtain the at least one frame of raw image.

In some examples, the one or more processors are further caused to: determine positions of invalid pixel values based on the at least some frames of to-be-detected images, and determine the invalid pixel values in the at least one frame of initial raw image based on the positions of invalid pixel values.

In some examples, the at least one spectral light source includes n types of spectral light sources with different emission spectra, and the at least one frame of raw image includes n frames of raw images respectively corresponding to the n types of spectral light sources, where n is an integer greater than or equal to 2. The n types of spectral light sources include a first spectral light source and a second spectral light source, the emission spectrum of the first spectral light source includes a wavelength of 850 nm, and the dominant wavelength of the emission spectrum of the second spectral light source is a wavelength other than 850 nm. The one or more processors are further caused to generate n frames of chromaticity maps respectively corresponding to the n frames of raw images, acquire weights of the n types of spectral light sources, and generate the one frame of representative chromaticity map based on the weights of the n types of spectral light sources and the n frames of chromaticity maps, wherein the weight of the first spectral light source is higher than the weight of the second spectral light source.

In some examples, the one or more processors are further caused to: obtain one frame of initial representative chromaticity map based on the at least one frame of raw image; acquire the current ambient temperature; determine a chromaticity compensation value based on the current ambient temperature from chromaticity compensation values corresponding to different temperatures; and determine the representative chromaticity map of the coffee particles based on the initial representative chromaticity values in the initial representative chromaticity map of the coffee particles and the chromaticity compensation value.

In some examples, the one or more processors are further caused to: for a k-th type of spectral light source, acquire a basis function corresponding to the k-th type of spectral light source, where the basis function is a relational function between raw pixel values collected under illumination of the k-th type of spectral light source and corresponding chromaticity values, and k is any integer from 1 to n; acquire raw pixel values in a k-th frame of raw image, the k-th frame of raw image being the raw image collected when the coffee particles are illuminated by the k-th type of spectral light source; and acquire corresponding chromaticity values based on the raw pixel values in the k-th frame of raw image and the basis function corresponding to the k-th type of spectral light source, so as to obtain the chromaticity map corresponding to the k-th frame of raw image.

In some examples, the one or more processors are further caused to: acquire at least one frame of initial raw image; acquire a brightness compensation function; determine a raw pixel compensation value for a pixel position based on the brightness compensation function and the pixel position in the initial raw image; and compensate the raw pixel value at the pixel position in the at least one frame of initial raw image based on the raw pixel compensation value for the pixel position.

In some examples, the one or more processors are further caused to acquire the representative chromaticity distribution of the representative chromaticity map; and the device further comprises a display configured to display the representative chromaticity distribution and the overall chromaticity value.

In some examples, the device further comprises a bearing surface for carrying the coffee particles, an illumination light source, and a backlight source. The vibration source is disposed on one side of the bearing surface. The illumination light source is disposed on the side of the bearing surface where the coffee particles are carried, and the backlight source is disposed on the other side of the bearing surface. The camera is configured to capture the to-be-detected image of the coffee particles on the bearing surface when the illumination light source and the backlight source emit light beams.

In some examples, the bearing surface is a first light homogenizing film. The device further comprises a second light homogenizing film and a light guide plate disposed between the vibration source and the first light homogenizing film, wherein the first light homogenizing film, the light guide plate, and the second light homogenizing film are arranged sequentially side by side, the light guide plate is located in a sealed space enclosed by the first light homogenizing film and the second light homogenizing film, and the backlight source is disposed at the periphery of the light guide plate.

A second aspect of the present application provides a method for analyzing coffee particles, the method comprising:
controlling a vibration source to drive coffee particles to vibrate at least twice, respectively capturing images of the coffee particles after the at least two vibrations, obtaining a to-be-detected image set containing coffee particles with different distributions;
respectively acquiring initial recognition information of coffee particles in the images to be detected in each to-be-detected image of the to-be-detected image set; and
determining final recognition information of the coffee particles according to the initial recognition information of coffee particles in at least some frames of to-be-detected images in the to-be-detected image set.

In some examples, controlling the vibration source to drive the coffee particles to vibrate at least twice and respectively capturing images of the coffee particles after the at least two vibrations to obtain a to-be-detected image set containing coffee particles with different distributions includes the following steps: 1. controlling the vibration source to drive the coffee particles to vibrate in a first driving mode, so as to obtain coffee particles with a first distribution; 2. capturing an image of the coffee particles with the first distribution, obtaining a first to-be-detected image; 3. controlling the vibration source to drive the coffee particles with the first distribution to vibrate in a second driving mode, obtaining coffee particles with a second distribution; and 4. capturing an image of the coffee particles with the second distribution to obtain a second to-be-detected image.

In some examples, the first driving mode and the second driving mode are different; controlling the vibration source to drive the coffee particles with the first distribution to vibrate in the second driving mode, and then further comprising: controlling the vibration source to drive the coffee particles to vibrate in the second driving mode at least once; capturing images of the coffee particles after each driving in the second driving mode, to obtain at least one frame of image to be detected.

In some examples, at least one of the following items is different between the first driving mode and the second driving mode: vibration frequency, vibration amplitude, vibration duration, and vibration area.

In some examples, the vibration frequency in the first driving mode is a resonance frequency, while the vibration frequency in the second driving mode is lower or higher than the resonance frequency; and/or the vibration amplitude in the first driving mode is greater than that in the second driving mode.

In some examples, the vibration amplitude in the first driving mode is greater than that in the second driving mode, and the vibration duration in the second driving mode is longer than that in the first driving mode.

In some examples, the second driving mode is determined based on the first to-be-detected image.

In some examples, the method further includes: determining at least one of vibration frequency, vibration amplitude, vibration duration, or vibration area in the second driving mode based on the initial recognition information of coffee particles in the first to-be-detected image; the initial recognition information of coffee particles in the first to-be-detected image includes the quantity and/or area of coffee particles in the first to-be-detected image.

In some examples, the initial recognition information of coffee particles in the to-be-detected image includes the quantity of coffee particles. The method further includes: determining a change in the quantity of coffee particles based on the quantity of coffee particles in at least one frame of to-be-detected image prior to the first to-be-detected image, the first to-be-detected image, and the second to-be-detected image; and determining a driving mode of the vibration source subsequent to the second to-be-detected image based on the quantity change.

In some examples, determining the driving mode of the vibration source subsequent to the second to-be-detected image based on the quantity change includes at least one of the following: when the quantity change is a decrease in quantity or the change value is less than a threshold, stopping the next driving of the coffee particles by the vibration source or continuing to drive the coffee particles in the second driving mode for the next time; or when the quantity change is an increase in quantity and the change value is greater than the threshold, continuing to drive the coffee particles in the first driving mode for the next time.

In some examples, the quantity of coffee particles in at least one frame of to-be-detected image prior to the first to-be-detected image, the first to-be-detected image, and the second to-be-detected image refers to the quantity of particles whose area exceeds a preset critical value in the at least one frame of to-be-detected image prior to the first to-be-detected image, the first to-be-detected image, and the second to-be-detected image.

In some examples, the initial recognition information includes at least one of the particle size, quantity, area, volume, mass, and chromaticity of the coffee particles; and/or at least one of the quantity, area, volume, mass, and chromaticity of tiny coffee particles. The tiny coffee particles refer to coffee particles with a particle size smaller than a first preset particle size, or a particle size smaller than the first preset particle size and larger than a preset critical value. In some examples, the initial recognition information further includes: at least one of the quantity distribution, area distribution, volume distribution, mass distribution, and chromaticity distribution of the coffee particles in different particle size ranges; and/or information on the proportion of the tiny coffee particles among all coffee particles.

In some examples, the final recognition information includes at least one of the final quantity distribution, final area distribution, final volume distribution, final mass distribution, and final chromaticity distribution of the coffee particles in different particle size ranges. The method further includes: displaying at least one of the final quantity distribution, final area distribution, final volume distribution, final mass distribution, and final chromaticity distribution of the coffee particles in different particle size ranges on an interactive interface.

In some examples, the initial recognition information of coffee particles includes the particle size of the coffee particles. Before determining the final recognition information of the coffee particles based on the initial recognition information of coffee particles in at least some frames of to-be-detected images in the to-be-detected image set, the method further includes: obtaining a distortion function, where the distortion function indicates particle size compensation values at multiple pixel positions; and performing distortion correction on the particle size of at least some coffee particles in the to-be-detected images of the to-be-detected image set according to the pixel positions of the coffee particles and the corresponding particle size compensation values, so as to obtain the distortion-corrected particle size of the coffee particles. In addition, determining the final recognition information of the coffee particles based on the initial recognition information of coffee particles in at least some frames of to-be-detected images in the to-be-detected image set includes: determining the final recognition information of the coffee particles based on the distortion-corrected particle size of coffee particles in at least some frames of to-be-detected images in the to-be-detected image set.

In some examples, the initial recognition information of coffee particles includes the particle size of the coffee particles. Before determining the final recognition information of coffee particles based on the initial recognition information of coffee particles in at least some frames of to-be-detected images in the to-be-detected image set, the method further includes: 1. obtaining a particle size compensation function, where the particle size compensation function indicates particle size compensation values corresponding to multiple brightness levels, 2. respectively acquiring the brightness of the regions where coffee particles are located in at least some frames of to-be-detected images in the to-be-detected image set, and 3. for the at least some frames of to-be-detected images, compensating the particle size of coffee particles in the to-be-detected images according to the brightness of the regions where coffee particles are located in the to-be-detected images and the corresponding particle size compensation values, so as to obtain the compensated particle size of coffee particles. In addition, determining the final recognition information of coffee particles based on the initial recognition information of coffee particles in the to-be-detected images includes: determining the final recognition information of coffee particles based on the compensated particle size of coffee particles in at least some frames of to-be-detected images in the to-be-detected image set.

In some examples, the initial recognition information of coffee particles includes the particle size of the coffee particles. The method further includes: entering a calibration mode, where in the calibration mode, the following operations are performed: capturing an image of a calibration pattern-having a preset area and located at a preset position within the field of view-to obtain a calibration image; acquiring the number of pixels corresponding to the calibration pattern; and determining a calibration size corresponding to one pixel according to the preset area and the number of pixels. The step of respectively acquiring the initial recognition information of coffee particles in the to-be-detected images of the to-be-detected image set includes: respectively acquiring the number of pixels of coffee particles in the to-be-detected images of the to-be-detected image set; and determining the particle size of the coffee particles according to the calibration size and the number of pixels.

In some examples, the to-be-detected image is an image captured when coffee particles are illuminated by an illumination light source. The method further includes: 1. acquiring at least one frame of raw image, where the at least one frame of raw image includes raw pixel values of images captured when the coffee particles are illuminated by at least one spectral light source different from the illumination light source; 2. obtaining one frame of representative chromaticity map based on the at least one frame of raw image; and 3. determining the overall chromaticity value of the coffee particles based on the one frame of representative chromaticity map.

### BRIEF DESCRIPTION OF THE DRAWINGS

For a more complete understanding of this disclosure, and the advantages thereof, reference is now made to the following brief description taken in connection with the accompanying drawings, in which:

FIG. 1 is a schematic diagram of an embodiment of a method for analyzing coffee particles of the present application.

FIG. 2 is a schematic diagram of an embodiment of a particle size distribution histogram of coffee particles according to the present application.

FIG. 3 is a diagram illustrating another embodiment of a particle size distribution histogram of coffee particles according to the present application.

FIG. 4 is a schematic diagram of a quantity distribution histogram included in the final recognition information of the coffee particles displayed on the interactive interface.

FIGS. 5 and 6 are images to be detected of coffee particles in regions with different brightness levels.

FIG. 7 is a schematic diagram of an embodiment of the method for analyzing coffee particle of the present application.

FIG. 8 is a schematic diagram of an interactive interface of the present application.

FIG. 9 is a schematic diagram of a captured coffee particle image.

FIG. 10 is a schematic diagram of an embodiment of the preset calibration color chart of the present application.

FIG. 11 is an exemplary diagram of an embodiment of the device for analyzing coffee particles of the present application.

FIG. 12 is another example diagram of an embodiment of the device for analyzing coffee particles of the present application.

FIG. 13 is a schematic diagram of the structure of the vibration source, the bearing surface, and the backlight source in one embodiment of the device for analyzing coffee particles of the present application.

FIG. 14 is a schematic diagram of the to-be-detected image captured by the camera when only the illumination light source is turned on.

FIG. 15 is a schematic diagram of the to-be-detected image captured by the camera when both the illumination light source and the backlight source are both turned on.

### DETAILED DESCRIPTION

The embodiments of this disclosure are discussed in detail below. It should be appreciated, however, that the present disclosure provides many applicable inventive concepts that can be embodied in a wide variety of specific contexts. The specific embodiments disclosed are merely illustrative of specific ways to make and use the invention, and do not limit the scope of the invention. It should be understood that the term “and/or” used herein refers to and includes any or all possible combinations of one or more associated listed items. It should also be understood that although the terms “first”, “second”, “third”, etc. may be used in this application to describe various information, these information should not be limited to these terms. These terms are only used to distinguish the same type of information from each other. For example, without departing from the scope of the present application, the first information may also be referred to as the second information, and similarly, the second information may also be referred to as the first information. Thus, a feature defined as “first” or “second” may explicitly or implicitly include one or more of the features. In the description of this application, “plurality” means two or more, unless otherwise clearly and specifically defined.

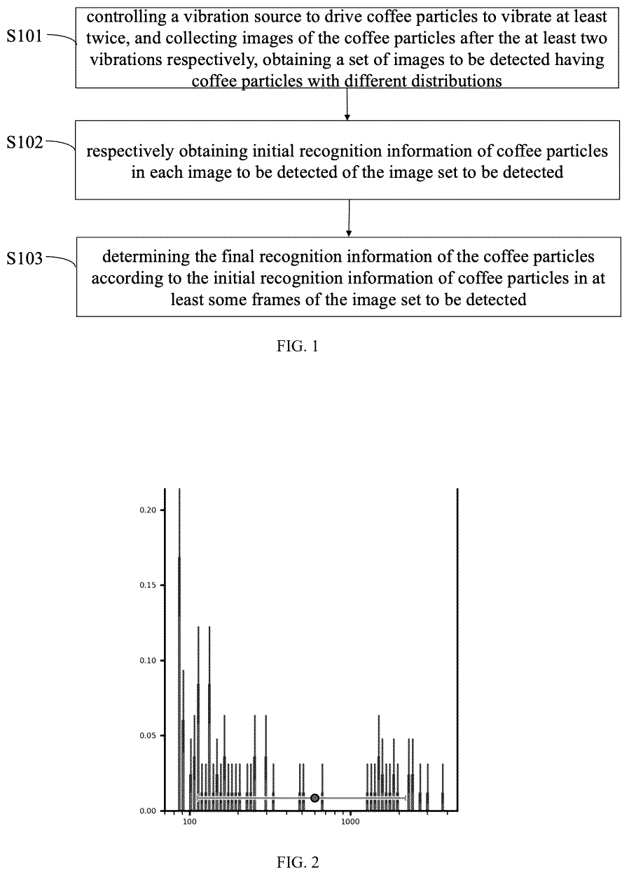

As shown in FIG. 1, FIG. 1 is a schematic diagram of an embodiment of a method for analyzing coffee particles of the present application. The method for analyzing coffee particles comprises:

Step S101, controlling a vibration source to drive coffee particles to vibrate at least twice, and collecting images of the coffee particles after the at least two vibrations respectively, obtaining a set of images to be detected having coffee particles with different distributions.

In some examples, coffee particles may refer to coffee beans or coffee powder or coffee particles of other shapes and particle sizes. In some examples, the method for analyzing coffee particle of the present application is applied to a device for analyzing coffee particle. In one example, the device for analyzing coffee particle is provided with a camera for collecting images containing coffee particles to obtain at least one images to be detected. In some examples, the camera includes a photosensitive array. The photosensitive array may include a charge-coupled device (CCD) or complementary metal oxide semiconductor (CMOS). In some examples, a bearing surface for bearing coffee particles is further provided in the device for analyzing coffee particles. For example, a sample tray is further provided in the device for analyzing coffee particles, and the bearing surface is the bottom surface of the sample tray. The photosensitive area array is configured to collect images of coffee particles located in the sample tray, obtaining multiple frames of images to be detected, and the image set to be detected includes the multiple frames of images to be detected. In some examples, a light source is further provided in the device for analyzing coffee particles, and the light source is configured to irradiate the coffee particles at least when the photosensitive area array images the coffee particles.

In some examples, a vibration source is disposed on the side of the bearing surface facing away from the coffee particles, and the vibration source is configured to drive the bearing surface to vibrate, thereby driving the coffee particles on the bearing surface to vibrate and changing the distribution of the coffee particles. In some examples, before capturing each frame of the to-be-detected image of the coffee particles, the vibration source is controlled to drive the coffee particles to vibrate.

In some examples, the bearing surface includes a vibration membrane, and the coffee particles are carried on the vibration membrane. The vibration membrane is driven to vibrate, thereby driving the coffee particles to vibrate. In some examples, the vibration source includes a power amplifier configured to emit sound waves to drive the vibration membrane to vibrate. In some examples, the vibration source includes a source configured to emit other mechanical waves, such as water waves and rope waves. In some examples, the vibration source drives the coffee particles to vibrate through other means (e.g., mechanical impact) rather than by emitting mechanical waves. In one example, the vibration source includes an electromagnetic coil, an electrode, an air hammer, or a spring connected to the bearing surface, which drives the coffee particles on the bearing surface to vibrate by impacting the bearing surface. In one example, the vibration source includes at least two linear vibration sources with different directions. The vibration direction and dispersion degree of the coffee particles can be controlled by adjusting the frequency of the transverse wave and longitudinal wave of each linear vibration source. In one example, multiple vibration sources may be disposed on the back of the bearing surface, in a Cartesian coordinate system centered at the center of the bearing surface. The multiple vibration sources may include three vibration sources whose vibration directions are parallel to the X, Y, and Z coordinate axes of the Cartesian coordinate system, respectively. In one example, voice coil motors with a vibration direction perpendicular to the bearing surface may be disposed at the four corners of the bearing surface. The distribution of the coffee particles can be changed by adjusting the amplitude and frequency of the voice coil motors and utilizing the principles of resonance and coherent wave interference.

In one example, the vibration source is controlled to drive the coffee particles to vibrate in a first driving mode, resulting in coffee particles with a first distribution; at least one image of the coffee particles with the first distribution is captured to obtain a first to-be-detected image. The vibration source is further controlled to drive the coffee particles with the first distribution to vibrate in a second driving mode, resulting in coffee particles with a second distribution; at least one image of the coffee particles with the second distribution is captured to obtain a second to-be-detected image. The first driving mode and the second driving mode may be the same. For instance, the vibration source may be controlled to drive the coffee particles to vibrate multiple times in a fixed manner, and images of the coffee particles after each vibration stop are captured, so as to obtain multiple frames of to-be-detected images containing coffee particles with different distributions.

In some examples, the first driving mode and the second driving mode may be different. In some examples, at least one of the following parameters differs between the first driving mode and the second driving mode: vibration frequency, vibration amplitude, vibration duration, and vibration area. In some examples, the vibration frequency in the first driving mode is a resonant frequency, and the vibration frequency in the second driving mode is less than or greater than the resonant frequency; and/or, the vibration amplitude in the first driving mode is greater than the vibration amplitude in the second driving mode. Since the resonant frequency of the vibration source drives the vibration amplitude to a maximum or near-maximum, initially vibrating the coffee particles at the resonant frequency or with a larger vibration amplitude can better disperse the coffee particles, enabling the first to-be-detected image to more accurately measure the sizes of the coffee particles. For instance, before capturing the first to-be-detected image of the coffee particles, the coffee particles are driven to vibrate at the resonant frequency. Then, before capturing the second to-be-detected image, a smaller vibration amplitude is used to fine-tune the distribution of the coffee particles. Using such a first to-be-detected image and a second to-be-detected image can improve the accuracy of the coffee particle size analysis.

In some examples, the vibration amplitude in the first driving mode is greater than that in the second driving mode, while the vibration duration in the second driving mode is longer than that in the first driving mode. This allows the coffee particles to be fully dispersed with a shorter vibration duration and larger vibration amplitude before acquiring the first to-be-detected image. The particle distribution can then be fine-tuned through further dispersion with a longer vibration duration and smaller vibration amplitude, facilitating the acquisition of more size information about the coffee particles. For instance, the vibration duration in the first driving mode may range from 0.1 s to 0.5 s, while the vibration duration in the second driving mode, which is longer than that in the first driving mode, may range from 0.1 s to 1 s. In some examples, the vibration duration in the second driving mode may be the same as that in the first driving mode, or it may be shorter than that in the first driving mode.

In some examples, after the vibration source is controlled to drive the coffee particles with the first distribution to vibrate in the second driving mode, the vibration source is further controlled to drive the coffee particles to vibrate in the second driving mode at least once more. Images of the coffee particles are captured after each vibration in the second driving mode, so as to obtain at least one frame of to-be-detected image. In some examples, during the process of the vibration source driving the coffee particles in the second driving mode, the vibration source may continuously capture images of the coffee particles at preset intervals without stopping the vibration of the coffee particles, so as to obtain multiple frames of to-be-detected images.

Step S102, respectively obtaining initial recognition information of coffee particles in each image to be detected of the image set to be detected.

In some examples, the initial recognition information includes at least one of the particle size, particle quantity, particle area, particle volume, and particle mass. In some examples, the initial recognition information further includes the distribution of at least one of the particle size, particle quantity, particle area, particle volume, and particle mass.

There are various methods to obtain the particle size of coffee particles. For example, the area occupied by the coffee particles in the image to be detected is measured, and this area is converted to the diameter of a circle with an equal area, which is then taken as the initial particle size of the coffee particles. In the prior art, the analysis of coffee particle size mostly adopts the powder sieving method or laser scattering method. The powder sieving method is low in cost but low in work efficiency, while the laser scattering method is high in work efficiency but expensive. Compared with these two methods, the image analysis method has the advantages of fast recognition speed, high analysis efficiency and low cost.

When detecting the area of coffee particles, the size of coffee particles in the image to be detected can be identified using a machine learning method. The coffee particles can be labeled as adherent or non-adherent, and/or the silver skin in the coffee particles can be labeled, thereby improving the recognition efficiency and accuracy of the coffee particle size. The volume of the coffee particles can be calculated based on their particle size and/or area. The mass of the coffee particles can be calculated based on their volume and the preset density of the coffee particles.

In some examples, the initial recognition information further includes distribution information of coffee particles in different particle size intervals. Such distribution information may include the number distribution, area distribution, volume distribution, mass distribution, and/or chromaticity distribution of coffee particles in different particle size intervals. For example, 9 particle size intervals are preset: 200 μm-300 μm, 300 μm-425 μm, 425 μm-600 μm, 600 μm-850 μm, 850 μm-1180 μm, 1180 μm-1400 μm, 1400 μm-1700 μm, 1700 μm-2360 μm, and 2360 μm-2500 μm. The number of preset particle size intervals and the length of each interval can be set in other ways, which are not limited herein. After determining the particle size of each coffee particle in the image to be detected, the number distribution of coffee particles in multiple preset particle size intervals can be the sum of the counts in each preset particle size interval, or the ratio of the sum of the counts in each preset particle size interval to the total count in all preset particle size intervals. Similarly, the area/volume/mass distribution of coffee particles in multiple preset particle size intervals can be the sum of the area/volume/mass in each preset particle size interval, or the ratio of the sum of the area/volume/mass in each preset particle size interval to the total area/volume/mass in all preset particle size intervals. The chromaticity distribution of coffee particles in multiple preset particle size intervals can be the average or median of the chromaticity values in each preset particle size interval.

As shown in FIG. 2, which is a schematic diagram of an embodiment of a particle size distribution histogram of coffee particles according to the present application. Each bar in the histogram corresponds to a preset particle size interval; the abscissa represents the particle size corresponding to the preset particle size interval, and the ordinate represents the ratio of the number of coffee particles in the preset particle size interval to the total number of coffee particles in all preset particle size intervals.

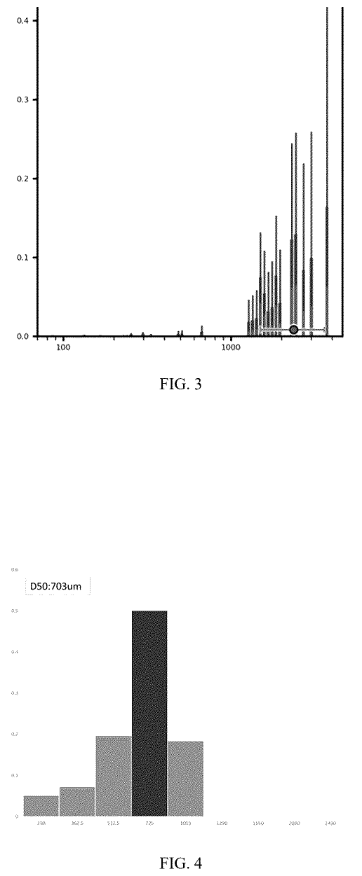

As shown in FIG. 3, which is a diagram illustrating another embodiment of a particle size distribution histogram of coffee particles according to the present application. Each bar in the histogram corresponds to a preset particle size interval; the abscissa represents the particle size corresponding to the preset particle size interval, and the ordinate represents the ratio of the total area of coffee particles in the preset particle size interval to the total area of coffee particles in all preset particle size intervals.

In one example, the initial recognition information further includes at least one of the count, area, volume, and mass of tiny coffee particles. The tiny coffee particles refer to coffee particles with a particle size smaller than a first preset particle size, or those with a particle size smaller than the first preset particle size but larger than a preset critical value. The first preset particle size is smaller than the particle sizes in the multiple preset particle size intervals. For example, tiny coffee particles are those with a particle size smaller than 200 μm. During coffee grinding, the variation in particle size depends on the requirements of different brewing methods. However, it is generally desirable to obtain coffee particles with higher uniformity, and tiny coffee particles are those generated during grinding that fall outside the expected particle size range. Obtaining information about tiny coffee particles can provide users with a reference for evaluating the grinding effect. For instance, if the count or area of tiny coffee particles exceeds a certain value, or if their count ratio or area ratio exceeds a certain value, it indicates poor grinding performance.

In some examples, the initial recognition information further includes the proportion of tiny coffee particles relative to all coffee particles. Such proportion information may be the ratio of the total count, total area, total volume, or total mass of tiny coffee particles to that of all coffee particles.

In some examples, before obtaining the initial recognition information of coffee particles in the image to be detected, it is also necessary to determine whether the coffee particles are coffee powder or coffee beans. Specifically, this determination can be made through user input, or by analyzing the image to be detected—for example, by obtaining the gradient of the image to be detected and determining whether the coffee particles are coffee powder or coffee beans based on the gradient classification result.

Step S103, determining the final recognition information of the coffee particles according to the initial recognition information of coffee particles in at least some frames of the image set to be detected.

For example, the final recognition information of the coffee particles can be obtained by calculating a weighted average of the initial recognition information of the coffee particles in all frames of the image to be detected within the image set to be detected. This helps reduce measurement errors and improve measurement accuracy. In some examples, the final recognition information is the initial recognition information derived from one frame of to-be-detected image selected from the to-be-detected image set. The selected one frame of to-be-detected image can be the one with the largest number of coffee particles in the to-be-detected image set. In some examples, it is also determined whether each frame of the image to be detected in the image set to be detected is valid, and the final recognition information of the coffee particles can be obtained only based on the initial recognition information of the coffee particles in valid images to be detected. There are various methods for determining the validity of an image to be detected. In one example, an image to be detected is determined to be invalid when there is severe adhesion of coffee particles in it. For instance, the area of the coffee particles in the image to be detected is measured. If the area of the coffee particles exceeds a first threshold, or if the number of coffee particles with an area exceeding a second threshold or the area ratio exceeds a preset ratio, the frame of the image to be detected is determined to be invalid. The first threshold and the second threshold may be the same or different.

When performing a weighted average calculation on the initial recognition information of coffee particles in at least some frames to be detected within the image set to be detected, the distribution information of each frame (among the at least some frames to be detected) across multiple different particle size intervals is obtained, and a weighted average calculation is performed on such distribution information. For instance, for each particle size interval, at least one parameter among the count, area, volume, and mass from multiple frames to be detected is obtained, and a weighted average calculation is performed on the same type of parameters from the multiple frames. In some examples, for tiny coffee particles, at least one parameter among the count, area, volume, and mass of tiny coffee particles from multiple frames to be detected is obtained, and a weighted average calculation is performed on the same type of parameters from the multiple frames.

In some examples, the weight values for each frame of the image to be detected are the same, and the same type of recognition information from multiple frames of the image to be detected are averaged. In some examples, the ratio of the total count in each particle size interval to the total count in all particle size intervals, the ratio of the total area in each particle size interval to the total area in all particle size intervals, the ratio of the total volume in each particle size interval to the total volume in all particle size intervals, or the ratio of the total mass in each particle size interval to the total mass in all particle size intervals may also be obtained.

In some examples, the final recognition information of the coffee particles is also displayed via an interactive interface. As shown in FIG. 4, which is a schematic diagram of a quantity distribution histogram included in the final recognition information of the coffee particles displayed on the interactive interface. The final recognition information includes the percentages of the total number of coffee particles in each of the 9 preset particle size intervals relative to the total number of coffee particles in all particle size intervals. The x-axis of FIG. 4 represents the median of each of the 9 preset particle size intervals, and the y-axis represents the ratio of the number of coffee particles in each particle size interval to the total number of coffee particles in all particle size intervals.

In some examples, the particle size interval containing the median particle size of the coffee particles is also displayed via the interactive interface. The median particle size refers to the particle size corresponding to the point where the cumulative particle size distribution percentage of the coffee particles reaches 50%. Its physical meaning is that particles with a particle size larger than the median particle size account for 50%, and those with a smaller particle size also account for 50%. As shown in FIG. 4, for example, if the median particle size falls within the 600 μm-850 μm interval, the number ratio of this interval is displayed in a different color or pattern from other intervals to indicate that it contains the median particle size.

In the particle size analysis of coffee particles, ensuring their discrete distribution is a prerequisite for improving measurement accuracy. If a large number of coffee particles adhere to one another, the particle size measurement result will be excessively large. In the embodiments of the present application, a vibration source drives the coffee particles to vibrate at least twice to disperse the adherent particles and alter their distribution. By acquiring recognition information from multiple frames of images showing coffee particles in different distributions to determine the final recognition information, the accuracy of coffee particle analysis can be improved.

In some examples, when controlling the vibration source to drive the coffee particles to vibrate multiple times, the driving mode for at least one vibration is determined based on one or more most recently captured images corresponding to that vibration. For example, the second driving mode is determined based on the first image to be detected. For example, the adhesion degree of coffee particles in the first image to be detected is obtained, and at least one parameter-vibration frequency, vibration amplitude, vibration duration, or vibration area-in the second driving mode is determined according to this adhesion degree.

In some examples, the adhesion degree is determined based on the quantity and/or area of the coffee particles in the first image to be detected. For instance, for a partial area or the entire area of the first image to be detected, the quantity and total area of the coffee particles within the area are detected, and when the ratio of the total area to the quantity in the area falls into different ranges, corresponding adhesion degrees are assigned. Alternatively, the area of each coffee particle in the area is detected, and when the area of the coffee particles falls into different ranges, or when the quantity of coffee particles with an area greater than the preset area falls into different ranges, corresponding adhesion degrees are assigned. In some examples, different adhesion degrees correspond to different vibration frequencies, vibration amplitudes, or vibration durations. After determining the adhesion degree of the coffee particles in the first image to be detected, the corresponding vibration frequency, amplitude, or duration is adopted as the parameter for the second driving mode based on the adhesion degree. In cases where the vibration source is capable of driving vibrations in different regions respectively, the vibration frequency, amplitude, or duration corresponding to each region can also be determined based on the adhesion degree of the coffee particles in that region.

In some examples, for regions with a relatively high adhesion degree, the second driving mode employs at least one of a higher vibration frequency, a larger vibration amplitude, or a longer vibration duration compared to the first driving mode. In some examples, only one of these parameters may be increased, while the others use default values. For instance, when determining the second driving mode based on the first image to be detected, both the vibration frequency and amplitude use default values, and the vibration duration is determined according to the adhesion degree of the coffee particles in the first image to be detected.

In some examples, the first image to be detected is fixedly divided into multiple regions in a preset manner-for example, evenly divided into four regions. The division of each region can be coordinated with the control precision of the vibration source, enabling the vibration source to vibrate different regions individually. For example, multiple vibration sources can be used to vibrate different regions separately, or the vibration position of a single vibration source can be moved to target different regions. For each region in the first image to be detected, the adhesion degree can be determined using the method described above. Regions with an adhesion degree higher than a preset threshold can be designated as the vibration target regions in the second driving mode. In other examples, the first image to be detected may not be fixedly divided into regions in a preset manner. In cases where the vibration source can control its driving position, after detecting a position with a coffee particle adhesion degree higher than the preset threshold, the vibration source can be driven with that position as the center of the vibration region.

In some examples, the adhesion degree of the coffee particles in the first image to be detected is obtained, and the vibration frequency in the second driving mode is determined based on the adhesion degree. The adhesion state of the coffee particles in the first image to be detected can be determined according to the adhesion degree, and the corresponding vibration frequency is then determined—for example, the higher the adhesion degree, the higher the vibration frequency.

In some examples, the initial recognition information of coffee particles in the image to be detected includes the quantity of coffee particles. After acquiring the initial recognition information, the quantity change information of the coffee particles is determined based on the quantity of coffee particles in at least one frame of image to be detected captured prior to the first image to be detected, the first image to be detected, and the second image to be detected. The driving mode of the vibration source subsequent to the second image to be detected is then determined according to this quantity change information. In some examples, if the quantity change information indicates that, in consecutive preset frames of images to be detected, the quantity of coffee particles decreases in each frame decreases, or the absolute value of the quantity change is less than a threshold, the vibration source either stops driving the coffee particles next time, or drives them in a mode where the vibration amplitude, vibration frequency, or vibration duration is less than the respective preset value. In some examples, if the quantity change information indicates that, in consecutive preset frames of images to be detected, the quantity of coffee particles increases in each frame and the change value exceeds the threshold, the coffee particles are driven in a mode where the vibration amplitude, vibration frequency, or vibration duration is greater than the respective preset value.

In some embodiments, the vibration source may be configured with only two distinct modes: a first driving mode and a second driving mode. The vibration frequency in the first driving mode is a resonant frequency, while the vibration frequency in the second driving mode is either lower or higher than the resonant frequency; and/or the vibration amplitude in the first driving mode is greater than that in the second driving mode. Before acquiring the first image to be detected of the coffee particles, the coffee particles are driven to vibrate in the first driving mode to disperse them. After acquiring the first image to be detected, the first driving mode may continue to be used to drive the coffee particles to vibrate before at least two subsequent acquisitions of images to be detected. In some examples, the second driving mode may alternatively be used to drive the coffee particles to vibrate before at least two subsequent acquisitions of images to be detected. After acquiring at least three frames of images to be detected, the change in the quantity of coffee particles is determined based on the quantity in each frame. If, in consecutive preset frames of images to be detected, the quantity of coffee particles decreases in each frame or the absolute value of the quantity change is less than a threshold, the vibration source either stops driving the coffee particles next time or continues to drive them in the second driving mode. In some examples, if the quantity shows an increasing trend and the change value exceeds the threshold, the next driving of the coffee particles is continued in the first driving mode.

In some examples, the initial recognition information of the coffee particles includes their particle sizes. And prior to step S103, a distortion function is also acquired, which indicates particle size compensation values at multiple pixel positions. The particle sizes of at least some of the coffee particles in the images to be detected (from the image set to be detected) are subjected to distortion correction based on the pixel positions of the coffee particles and the corresponding particle size compensation values, so as to obtain the distortion-corrected particle sizes of the coffee particles. In step S103, the final recognition information of the coffee particles is determined according to the distortion-corrected particle sizes from at least some frames of the images to be detected in the image set to be detected. Due to the imaging distortion of the camera, errors may occur in the particle size measurement of coffee particles at the edge of the image, and such errors can be reduced by correcting via the distortion function. The distortion function can be obtained and stored by calibrating the equipment before it leaves the factory. In some examples, during the calibration process, a checkerboard pattern can be imaged using a photosensitive array in the coffee particle analysis equipment, thereby fitting the distortion coefficient for each pixel position-that is, the distortion function.

In some examples, the distortion function is a relationship curve between the distance from a pixel position to the center position of the image and the difference between the actual particle size at that pixel position and the calculated particle size. In particle size analysis, after obtaining the distance from the pixel position of a coffee particle to the center of the image, the difference between the calculated particle size and the actual particle size can be derived based on this distance and the distortion function. By compensating the calculated particle size with this difference, distortion correction is achieved, resulting in a more accurate particle size measurement result.

In some examples, the initial recognition information of the coffee particles includes their particle sizes. And prior to step S103, a particle size compensation function is acquired, which indicates particle size compensation values under multiple brightness conditions. Additionally, the brightness of the regions where the coffee particles are located in at least some frames of the images to be detected (from the image set to be detected) is obtained respectively. For these at least some frames of images to be detected, the particle sizes of the coffee particles therein are compensated according to the brightness of their respective located regions and the corresponding particle size compensation values, so as to obtain the compensated particle sizes of the coffee particles. In step S103, the final recognition information of the coffee particles is determined based on the compensated particle sizes of the coffee particles in at least some frames of the images to be detected in the image set to be detected. In particle size analysis, uneven brightness may be caused by non-uniform illumination sources or different backgrounds of the bearing surface. Such uneven brightness may lead to inconsistent particle size calculation results for the same coffee particles.

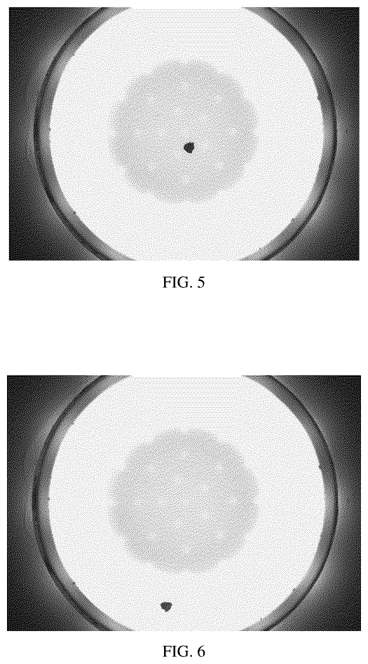

For example, as shown in FIGS. 5 and 6, which are images to be detected of coffee particles in regions with different brightness levels. In FIG. 5, the coffee particles are located in region 701 with moderate brightness, and their edges are relatively clear. In FIG. 6, the coffee particles are located in region 601 with excessive brightness, which may cause overexposure of the coffee particles' edges. This appears as edge erosion of the coffee particles in the image to be detected, leading to measurement errors in particle size analysis. Therefore, during the pre-factory calibration process, by analyzing the particle sizes of the same coffee particles in regions with different brightness levels, the influence of varying brightness on the particle size calculation of coffee particles can be obtained and stored. In actual particle size analysis, compensation and calibration are performed on the particle sizes of coffee particles in regions with different brightness levels to improve measurement accuracy.

In some examples, the initial recognition information of the coffee particles includes their particle sizes. The method for analyzing coffee particle of the present application further includes entering a calibration mode, wherein the calibration mode involves the following steps.

Step 1, capturing an image of a calibration pattern-with a preset area and located at a preset position within the field of view-to obtain a calibration image.

Step 2, acquiring the number of pixels corresponding to the calibration pattern.

Step 3, determining a calibration size per pixel based on the preset area and the number of pixels.

When determining the particle sizes of the coffee particles in step S102, the number of pixels occupied by the coffee particles in each image to be detected (from the image set to be detected) is acquired respectively; the particle sizes of the coffee particles are then determined based on the calibration size per pixel and the number of pixels.

In practical applications, the calibration mode may be entered upon user triggering. Alternatively, the system may automatically enter the calibration mode at preset intervals by default to obtain the latest calibration size, or the user may set the triggering method for the calibration mode.

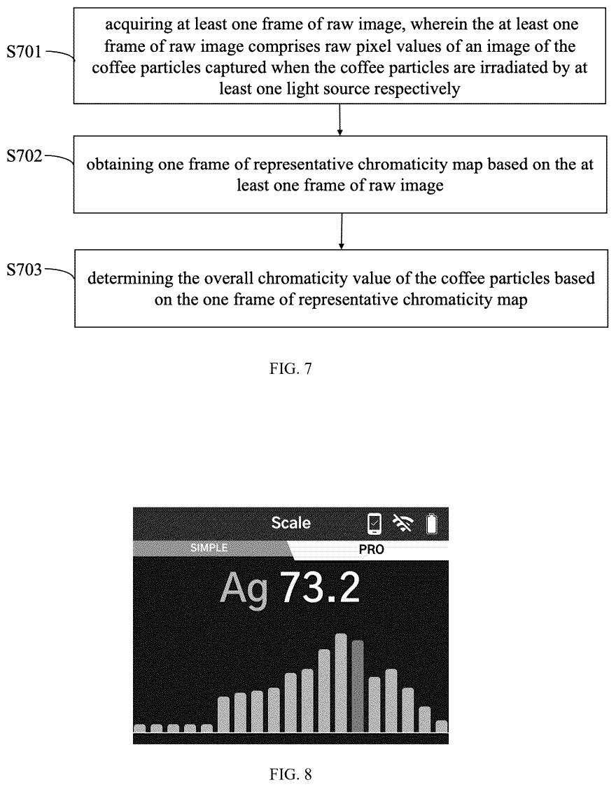

In some examples, the method for analyzing coffee particles of the present application not only includes analyzing the quantity, particle size, area, volume, or mass of coffee particles, but further includes analyzing their chromaticity. As shown in FIG. 7, which is a schematic diagram of an embodiment of the method for analyzing coffee particle of the present application. In some examples, the method for analyzing coffee particle in this embodiment further includes the following steps.

Step S701, acquiring at least one frame of raw image, wherein the at least one frame of raw image comprises raw pixel values of an image of the coffee particles captured when the coffee particles are irradiated by at least one light source respectively.

In some examples, the at least one light source includes a light source whose emission spectrum covers a wavelength of 850 nm. In some examples, the at least one light source is different from the light source in the aforementioned device for analyzing coffee particle. To facilitate distinguishing between the at least one light source and the light source mentioned above, the former is hereinafter referred to as a “spectral light source”, and the light source mentioned above is hereinafter referred to as a “illumination light source”. In some examples, the illumination light source is configured to emit visible light, such as white light, and the spectral light source is configured to emit near-infrared light.

In some examples, “coffee particles” may refer to coffee beans, coffee powder, or coffee particles of other shapes and sizes. In one example, the device for analyzing coffee particle is equipped with only one spectral light source. In another example, the device for analyzing coffee particle is equipped with at least two spectral light sources with different emission spectra. In some examples, the at least two spectral light sources are configured to emit light beams with different wavelengths ranging from 500 nm to 1100 nm. Experimental results show that the reflection of coffee particles on spectra within this range is more conducive to calculating their chromaticity values. For instance, the device for analyzing coffee particle is equipped with at least some of six spectral light sources whose emission spectra correspond to wavelengths of 520 nm, 600 nm, 640 nm, 850 nm, 940 nm, and 1100 nm. Since coffee particles with the same roasting chromaticity exhibit different reflectivities to light beams of different spectra, multiple spectral light sources with different spectra irradiate the coffee particles, and the reflected light forms different raw images. By fusing the raw images corresponding to different spectral light sources through an algorithm to determine the chromaticity value of the coffee particles, the problem of poor stability in chromaticity value detection results caused by spectrum acquisition using a single light source can be effectively avoided.

In an example where the device for analyzing coffee particles is equipped with one spectral light source, the photosensitive array in the device is used to capture images of the coffee particles while the spectral light source irradiates them, thereby obtaining at least one frame of initial raw images. In an example where the device for analyzing coffee particles is equipped with n spectral light sources (where n≥2), the n spectral light sources are configured to irradiate the coffee particles at different times respectively. The photosensitive array is used to capture images of the coffee particles during irradiation by each spectral light source, so as to obtain at least n frames of initial raw images—wherein each of the n frames of initial raw images corresponds to one spectral light source.

Among them, one frame of raw image corresponding to each spectral light source may be a frame of initial raw image captured by the photosensitive array when the spectral light source irradiates the coffee particles, or a frame of raw image obtained by fusing multiple frames of initial raw images captured by the photosensitive array during the irradiation of the coffee particles by the spectral light source.

The initial raw image includes the raw pixel value of each pixel position, also referred to as raw data. Raw data refers to the original record of the signal level generated when the camera converts optical signals into electrical signals during the capture of coffee particle images. It is unprocessed raw data. Raw data is generally output in a specific order, such as GRBG, RGGB, BGGR, or GBRG—where R stands for red, G for green, and B for blue. There are three common formats for raw data: raw8, raw10, and raw12, which indicate that each pixel has 8-bit, 10-bit, and 12-bit data, respectively.

Step S702: obtaining one frame of representative chromaticity map based on the at least one frame of raw image.

The chromaticity values in the representative chromaticity map can have multiple meanings. For example, the chromaticity value may specifically be the “Agtron” which is roasting degree index of coffee particles, or other values that can reflect the chromaticity of coffee particles.

There are multiple methods for obtaining a representative chromaticity map based on the at least one frame of raw image. In an example where there is only one spectral light source (i.e., the at least one frame of raw image is specifically one frame of raw image), a basis function is acquired. The basis function may be pre-stored and is a function correlating the raw pixel values collected under the illumination of the spectral light source with the corresponding chromaticity values. The corresponding chromaticity values are obtained based on the raw pixel values in the raw image and the corresponding basis function, thereby generating the chromaticity map corresponding to the raw image.

In an example with n spectral light sources having different emission spectra (where n≥2)—i.e., the at least one frame of raw image specifically refers to n frames of raw images—a frame of representative chromaticity map can be obtained using a linear model method.

In the linear model method, for example, the n frames of raw images may first be fused into one frame of raw image, and then the corresponding chromaticity map is generated based on the fused raw image. In some examples, weights for the n spectral light sources may also be acquired, and the n frames of raw images are fused into one frame of raw image according to these weights—for instance, via weighted summation. In some examples, the linear model method may involve generating corresponding chromaticity maps from the n frames of raw images respectively to obtain n frames of chromaticity maps, which are then fused into one frame of representative chromaticity map. In some examples, weights for the n spectral light sources may be acquired, and the one frame of representative chromaticity map is generated based on these weights and the n frames of chromaticity maps—for example, by fusing them via weighted summation.

In one example, five spectral light sources with different emission spectra are provided, with corresponding weights a₁, a₂, a₃, a₄, and as respectively, and five frames of chromaticity maps are obtained corresponding to these five spectral light sources. For the chromaticity values at the same pixel position across the five chromaticity maps—denoted as Ag₁, Ag₂, Ag₃, Ag₄, and Ag₅ respectively—the representative chromaticity value at this pixel position is calculated as: representative chromaticity value=a₁×Ag₁+a₂×Ag₂+a₃×Ag₃+a₄×Ag₄+a₅×Ag₅.

There are multiple methods for generating n corresponding frames of chromaticity maps from n frames of raw images. For example, for the k-th spectral light source, a basis function corresponding to the k-th spectral light source is acquired. This basis function is a relational function between the raw pixel values collected under the irradiation of the k-th spectral light source and the corresponding chromaticity values (where k is any integer from 1 to n). The raw pixel values in the k-th frame of raw image (which is the raw image collected when the coffee particles are irradiated by the k-th spectral light source) are obtained. The corresponding chromaticity values are then derived based on the raw pixel values in the k-th frame of raw image and the basis function corresponding to the k-th spectral light source, thereby generating the chromaticity map corresponding to the k-th frame of raw image.

Step S703, determining the overall chromaticity value of the coffee particles based on the one frame of representative chromaticity map.

In some examples, a corresponding representative chromaticity distribution is obtained from the one frame of representative chromaticity map, where the representative chromaticity distribution includes the ratio of the number of pixels corresponding to different representative chromaticity values to the total number of pixels. In some examples, the representative chromaticity value with the highest ratio is used as the overall chromaticity value of the coffee particles. Alternatively, the weighted average of multiple representative chromaticity values with ratios exceeding a preset threshold is used as the overall chromaticity value, or the average value or median of the representative chromaticity distribution is adopted as the overall chromaticity value. In particular, when a weighted average is calculated for multiple chromaticity values with ratios exceeding the preset threshold, the weight of each chromaticity value can be determined based on its corresponding ratio. For example, the higher the ratio, the greater the weight assigned to the chromaticity value.

Compared with the prior art that calculates chromaticity values using grayscale images output by a photosensitive array, the embodiments of the present application acquire raw images captured directly by the photosensitive array—and the accuracy of raw images is much higher than that of grayscale images. Generally, the value range of a grayscale image is 0 to 255, while the upper limit of the value range of a raw image can reach three to four thousand or even higher. Calculating chromaticity values directly from raw images can greatly improve the accuracy of chromaticity values. Furthermore, there is no need for conversion using the grayscale values of grayscale images, which further enhances the accuracy of chromaticity values.

In some examples, after obtaining at least one frame of representative chromaticity map from the raw images and the overall chromaticity value of the coffee particles, at least one of the overall chromaticity value and the representative chromaticity distribution is displayed on an interactive interface. As shown in FIG. 8 (a schematic diagram of an interactive interface of the present application), the representative chromaticity distribution is displayed on the interactive interface in the form of a chromaticity histogram, specifically showing the sum of pixel areas in different representative chromaticity value intervals or the ratio of the sum of pixel areas to the total area. In addition, the overall chromaticity value is also displayed on the interactive interface, specifically “AG 73.2”. In some examples, the interactive interface also highlights the representative chromaticity value interval corresponding to the overall chromaticity value in the chromaticity histogram to indicate that the overall chromaticity value falls within this interval.

In step S702, the basis functions for spectral light sources of different spectra can be obtained through calibration and pre-stored; during calculation, the pre-stored weights are directly read for computation. There are multiple calibration methods. For example, for the first spectral light source, it can be used to illuminate a plurality of preset color charts with different chromaticity values, and the raw pixel values corresponding to each preset color chart are acquired. Since the chromaticity value of each preset color chart is known, the basis function corresponding to the first spectral light source can be fitted using the chromaticity values of the plurality of preset color charts and their corresponding raw pixel values. The calibration method for the basis functions of other spectral light sources can be similar to that of the first spectral light source.

In some examples, when acquiring the basis function for each spectral light source, the weight of the spectral light source is also determined according to the degree of linear correlation between the chromaticity values of the multiple preset color charts and the corresponding raw pixel values. In some examples, when setting the weights for spectral light sources of different spectra, a higher weight may be assigned to the spectral light source with a stronger linear correlation between raw data and chromaticity values. In some examples, the n spectral light sources include a first spectral light source and a second spectral light source, where the emission spectrum of the first spectral light source covers a wavelength of 850 nm, and the dominant wavelength of the emission spectrum of the second spectral light source is a wavelength other than 850 nm, and the weight of the first spectral light source is higher than that of the second spectral light source. Through numerous experiments, the inventors found that the linear correlation between raw data and chromaticity values—obtained by irradiating coffee particles with a spectral light source of 850 nm wavelength—is the strongest. Setting the first spectral light source to the highest weight helps improve calculation accuracy.

Since infrared spectroscopy is more sensitive to light-colored objects, even a slight color change in light-colored objects will cause a significant change in the raw data obtained by imaging. This results in low accuracy of chromaticity values when only infrared spectral light sources are used to image coffee particles. In the embodiments of the present application, imaging coffee particles using spectral light sources of different spectra to obtain raw images can provide multi-dimensional information for calculating the chromaticity value of coffee particles. By fusing this multi-dimensional information to calculate the chromaticity value of coffee particles, the calculation accuracy is improved.

In some examples, in step S702, a representative chromaticity map may also be obtained from the at least one frame of raw image using other methods. For example, the representative chromaticity map may be obtained via a Gaussian mixture model method. In this method, the Gaussian mixture model may be pre-stored in the device for analyzing coffee particle and acquired by reading the stored data. The Gaussian mixture model includes the probability density functions of m known

Gaussian models and the weights of the m Gaussian models (where the weights were initially unknown); the m Gaussian models correspond to the n-dimensional Gaussian distributions of the raw pixel values collected from each of the m preset color charts when irradiated by the n spectral light sources, and m is an integer greater than or equal to 2. For example, m may be 10, 12, 13, 15, or other integers. For example, the Gaussian mixture model can be expressed as:

$$p ( x ) = {\sum }_{k = 1}^{m} {π}_{k} N ( x \vert {μ}_{k} , {\sum }_{k} ) .$$

Wherein, πₖ represents the weight of the k-th Gaussian model, N(x|μₖ, Σₖ) represents the probability density function of the k-th Gaussian model, x represents the raw pixel value, and p(x) represents the Gaussian distribution of the raw pixel values in the raw image. In this model, N(x|μₖ, Σₖ) is known. N(x|μₖ, Σₖ) can be obtained by calibration, in which the n spectral light sources are used to illuminate the m preset color charts respectively to collect raw data, and the Gaussian distribution is obtained and stored based on the raw data.

After acquiring n frames of raw images, the Gaussian distribution of the raw pixel value at the first pixel position is obtained based on the n frames of raw images; the weights of the m Gaussian models are then acquired according to the Gaussian distribution of the raw pixel value at the first pixel position and the Gaussian mixture model. For example, for the first pixel position, an n-dimensional vector composed of the n raw pixel values (of the first pixel position) from the n frames of raw images is acquired as x, and the Gaussian distribution of the n raw pixel values is substituted into the aforementioned Gaussian mixture model as p(x). Since N(x|μₖ, Σₖ) is known, the weights nk of the m Gaussian models corresponding to the first pixel position can be derived from the Gaussian distribution p(x) and N(x|μₖ, Σₖ).

The representative chromaticity value at the first pixel position is obtained based on the weights of the m Gaussian distributions corresponding to the first pixel position and the chromaticity values of the m preset color charts. For example, the representative chromaticity value at the first pixel position can be calculated using the following formula:

$$A ( x ) = {\sum }_{k = 1}^{m} {π}_{k} {A}_{k} .$$

Wherein, Aₖ represents the chromaticity value of the preset color chart corresponding to the k-th Gaussian model, and the chromaticity value of each preset color chart is known. After substituting πₖ into the above formula, the representative chromaticity value at the first pixel position can be obtained. In this manner, the representative chromaticity values at multiple pixel positions are calculated respectively to obtain a representative chromaticity map.

In some examples, the n frames of raw images may be sent to a server, so that the server generates a representative chromaticity map corresponding to the n frames of raw images based on the n frames of raw images and a preset Gaussian mixture model; the device for analyzing coffee particle then receives the representative chromaticity map sent by the server. The method for generating the representative chromaticity map corresponding to the n frames of raw images based on the n frames of raw images and the preset Gaussian mixture model may refer to the above description and will not be repeated herein. By offloading the computation to the server, the computing power cost of the device for analyzing coffee particle can be reduced while maintaining the accuracy of chromaticity value calculation.

In practical applications, the inventors found that when the chromaticity value—specifically the Agtron value—is greater than a preset threshold, the relationship between the chromaticity value and the raw pixel value exhibits an obvious nonlinear characteristic. In the linear model method, linear deviation can be compensated for by weighted averaging the chromaticity responses at different wavelengths, thereby improving the accuracy of chromaticity value calculation. Herein, the preset threshold is an Agtron value ranging from 95 to 105; for example, Agtron 100. When the chromaticity value—specifically the Agtron value—is greater than the preset threshold, the distribution coupling degree of raw pixel values corresponding to different Agtron values at different wavelengths is relatively high. Fitting via the Gaussian mixture model can better distinguish each distribution. Therefore, in some examples, when obtaining one frame of representative chromaticity map from n frames of raw images, n frames of chromaticity maps are first generated from the n frames of raw images. If the area of the region (in the n frames of chromaticity maps) where the chromaticity value is greater than the preset threshold exceeds a preset area, or if the proportion of this area to the total area exceeds a preset proportion, the one frame of representative chromaticity map is obtained using the Gaussian mixture model method. In some examples, if the area of the region where the chromaticity value is greater than the preset threshold is not greater than the preset area, or if the proportion of this area is not greater than the preset proportion, the one frame of representative chromaticity map is obtained using the linear model method.

In some examples, the method for analyzing coffee particles of the present application further includes entering an automatic calibration mode. The following operations are performed in the automatic calibration mode.

Step 1: Acquire at least one frame of raw calibration image, where the at least one frame of raw calibration image includes raw pixel values of an image captured of a preset calibration color chart-when the preset calibration color chart at a preset position is irradiated by at least one spectral light source.

Step 2: Calculate the chromaticity value of the preset calibration color chart based on the raw pixel values in the at least one frame of raw calibration image.

Step 3: Calculate a chromaticity compensation value based on the chromaticity value calculated for the preset calibration color chart and the actual chromaticity value of the preset calibration color chart.

Among them, the preset calibration color chart at the preset position may mean that the user places the preset calibration color chart on the bearing surface, and then activates the spectral light source and the photosensitive array to capture images of the preset calibration color chart. In some examples, the preset calibration color chart may be fixed inside the device for analyzing coffee particle and positioned at a location (within the field of view of the photosensitive array) other than the bearing surface—for instance, fixed on the inner wall of the device for analyzing coffee particle—so that it lies within the field of view of the photosensitive array without blocking the bearing surface. After entering the automatic calibration mode, the chromaticity compensation value is obtained using the image area corresponding to the preset calibration color chart in the captured raw image. In some examples, the preset calibration color chart is fixed inside the device for analyzing coffee particles but outside the field of view of the photosensitive array; the device for analyzing coffee particles is further provided with a mechanical module, which is used to move the preset calibration color chart into the field of view of the photosensitive array after entering the automatic calibration mode, enabling the photosensitive array to capture the raw image of the preset calibration color chart.

In some examples, the aforementioned calibration pattern with a preset area may be located on the preset calibration color chart. As shown in FIG. 10 (a schematic diagram of an embodiment of the preset calibration color chart of the present application), the preset calibration color chart has a known chromaticity value, and is provided with a calibration pattern of a preset area (a circle is used as an example in FIG. 10). In this way, after acquiring the image of the preset calibration color chart, the circle can also be used to determine the calibration size corresponding to a single pixel.

In practical applications, due to factors such as the attenuation of the spectral light source, the finally calculated chromaticity value may differ from the actual chromaticity value. Therefore, the chromaticity compensation value is obtained through the automatic calibration mode, and when calculating the chromaticity value of coffee particles, the chromaticity compensation value is applied to the calculated chromaticity value of the coffee particles to improve calculation accuracy. For example, the following operations are performed in step S702.

Step 1: Obtain one frame of initial representative chromaticity map based on the at least one frame of raw image. The method for obtaining the initial representative chromaticity map may be the same as the aforementioned method for obtaining the representative chromaticity map.

Step 2: Determine the representative chromaticity map of the coffee particles based on the chromaticity values in the initial representative chromaticity map of the coffee particles and the chromaticity compensation value. For example, the representative chromaticity value of each pixel position (i.e., the representative chromaticity map) is obtained by adding or subtracting the chromaticity compensation value to/from the initial representative chromaticity value of each pixel position in the initial representative chromaticity map.

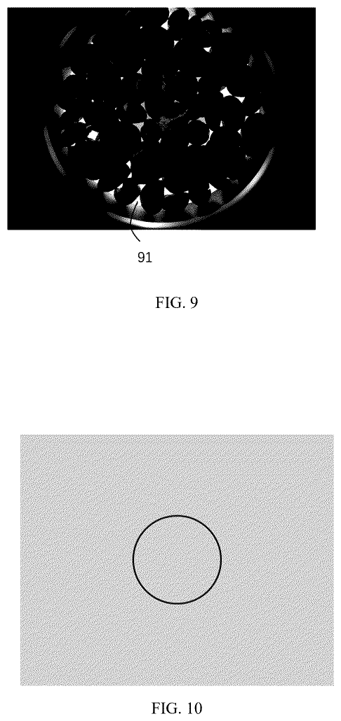

In practical applications, coffee particles may not occupy the entire raw image, resulting in some pixels that do not correspond to coffee particles. Calculating the chromaticity value of coffee particles based on these non-corresponding pixels will reduce the calculation accuracy. As shown in FIG. 9 (a schematic diagram of a captured coffee particle image), a partial pixel area 91 corresponding to the bearing surface can be observed in the captured image. Therefore, in some examples, when acquiring at least one frame of raw image in step S701, at least one frame of initial raw image is first acquired, and the at least one frame of raw image is then determined based on the at least one frame of initial raw image. Specifically: after acquiring the initial raw image, invalid pixel values are identified from the initial raw image-where the invalid pixel values refer to the pixel values corresponding to objects other than coffee particles in the initial raw image; and the invalid pixel values in the at least one frame of the initial raw image are removed to obtain the at least one frame of raw image. In this way, the representative chromaticity map obtained from the at least one frame of raw image in the subsequent step S702 can more accurately reflect the chromaticity values of coffee particles.

There are multiple methods for identifying valid pixel values or invalid pixel values. In some examples—where the raw image is an image of coffee particles placed on a bearing surface—the raw image contains invalid pixel values corresponding to the bottom of the bearing surface, as the coffee particles fail to cover the entire bearing surface. The presence of these invalid pixel values will affect the measurement of the chromaticity values of the coffee particles. The bearing surface can be configured to have a color (e.g., white) that is significantly different from that of the coffee particles, a texture that is significantly different from the surface texture of the coffee particles, or an easily recognizable pattern. In this way, invalid pixel values can be identified by detecting the color, texture, or pattern in the raw image. In some examples, the positions of valid pixel values or invalid pixel values are first identified based on the at least one of the image to be detected, and then be used to identify the valid pixel values or invalid pixel values in the raw image.

In some examples, there is no need to detect the color, texture, or pattern of the raw image. Due to the similarity among coffee particles, valid pixel values share certain commonalities and are significantly different from invalid pixel values. Thus, valid pixel values or invalid pixel values can be identified based on the differences and/or similarities of the raw data at each pixel position.

In some examples, when acquiring at least one frame of raw image in step S701, the following operations are performed: acquire at least one frame of initial raw image; acquire a brightness compensation function, and determine the raw pixel compensation value for a pixel position according to the brightness compensation function and the pixel position in the initial raw image; and compensate the raw pixel value at the pixel position in the at least one frame of initial raw image based on the raw pixel compensation value for the pixel position, so as to obtain the at least one frame of raw image.

In colorimetric analysis, the non-uniformity of the spectral light source may lead to inconsistent brightness across different areas. This, in turn, causes pixels with the same chromaticity value to exhibit different raw pixel values in areas of varying brightness, resulting in analysis errors. Therefore, during the calibration process of the coffee particle colorimetric analysis equipment prior to factory shipment, raw pixel value distributions corresponding to different brightness levels (at different pixel positions) can be obtained by analyzing a sample of coffee particles with a single and uniform chromaticity distribution across the entire field of view. The raw pixel compensation values for different pixel positions—i.e., the brightness compensation function—are then calculated. For example, the brightness compensation function may be the relationship between the distance from a pixel position to the image center and the raw pixel compensation value. In this manner, the brightness of pixels in different areas is compensated and calibrated algorithmically, thereby improving the measurement accuracy of colorimetric analysis.

In some examples, the bearing surface specifically refers to the bottom surface of a sample tray. The depth of the sample tray is set such that it can hold at least two layers of coffee particles, thereby preventing the bottom surface of the sample tray from appearing in the raw image when imaging the coffee particles.

In some examples, when determining the at least one frame of raw image based on the at least one frame of initial raw image in step S701, the method may further include: obtaining the distance distribution between the coffee particles and the camera; and compensating the pixel values in the at least one frame of initial raw image according to the distance distribution to obtain the at least one frame of raw image.

In practical applications, when the photosensitive array images coffee particles, variations in the distance between photosensitive array and the coffee particles will result in differences in the raw data contained in the captured raw image. These differences lead to inconsistent measurement benchmarks for the chromaticity values of different coffee particles in the raw image, thereby causing deviations in the measurement results. By compensating the pixel values in the at least one frame of raw image according to the distance distribution and performing calculations based on the compensated at least one frame of raw image, the accuracy of the chromaticity value measurement for coffee particles can be further improved.

There are multiple methods for obtaining the distance distribution between coffee particles and the camera. In some examples, the distance measurement module is controlled to emit a light beam toward the coffee particles and receive the return light reflected by the coffee particles; the distance distribution between the coffee particles and the camera is then obtained based on the emitted light beam and the return light. The raw image is divided into at least one region, and the distance distribution refers to the distance between the coffee particles and the camera in each region. It can be understood that the more regions divided, the finer the distance distribution.

In some examples, the distance measurement module is a laser distance measurement module disposed adjacent to the camera. It is configured to emit a laser beam toward the coffee particles, receive the laser beam reflected by the coffee particles, and measure the distance between the coffee particles and the camera based on the emitted and received laser beams. The measurement methods may include the pulse method, coherent method, or triangulation method, among others. Since the laser triangulation method has lower requirements for the laser distance measurement module and thus results in lower costs, the laser distance measurement module may adopt the laser triangulation method.

In some examples, the bearing surface refers to the bottom surface of the sample tray, and the camera is used to capture images of the coffee particles in the sample tray. The process is as follows: divide the image into at least one region; identify the target pixel region (corresponding to the bottom surface of the sample tray) in each region; and obtain the distance distribution between the coffee particles and the camera based on the proportion of the target pixel region in each region. Specifically, in a divided region: if the ratio of the area of the target pixel region (corresponding to the bottom surface of the sample tray) to the area of the region exceeds a certain value, it can be determined that the coffee particles in the region are only spread in a single layer in the sample tray. The distance between the coffee particles and the camera in this region can then be estimated based on the pre-calibrated height of the coffee particles and the pre-calibrated distance between the sample tray and the camera. If the proportion of the target pixel region (corresponding to the bottom surface of the sample tray) in the region is less than a certain value, it can be considered that the coffee particles in the region cover the sample tray; in this case, the pre-calibrated distance between the sample tray and the camera is used as the distance between the coffee particles and the camera in the region.

After determining the distance distribution between the coffee particles and the camera, the pixel values in the at least one frame of raw image are compensated according to the distance distribution, thereby adjusting the raw pixel values in the raw image to those corresponding to a uniform distance between all coffee particles and the camera. For example, based on the pre-calibrated correlation function between distance and raw pixel value, the raw pixel values in at least one region of the at least one frame of raw image can be compensated according to the acquired distance distribution, thus adjusting the raw pixel values in that region of the raw image to those corresponding to a preset distance between the coffee particles and the camera.

In some examples, a vibration source is further provided on the other side of the sample tray's bottom surface, configured to drive the bottom surface of the sample tray to vibrate, thereby evenly distributing the coffee particles in the sample tray. In some examples, before each acquisition of the raw image, the vibration source is used to vibrate the bottom surface of the sample tray to evenly distribute the coffee particles. This ensures that when the distance measuring module acquires the distance between the coffee particles and the camera, it is not necessary to acquire the distance distribution across different regions of the sample tray—only the distance at one location between the coffee particles and the camera needs to be acquired. When compensating the raw pixel values in the raw image, all regional raw pixel values in the raw image are uniformly adjusted, based on the acquired distance at that one location, to those corresponding to the preset distance between the coffee particles and the camera.

In some examples, in the scenario where the distance distribution between coffee particles and the camera is obtained, if it is detected that the distance distribution meets a preset condition, mechanical waves are emitted to the sample tray to vibrate it, thereby changing the distribution of coffee particles in the sample tray. The preset condition may be: when the difference between individual distances in the distance distribution between coffee particles and the camera exceeds a preset difference, mechanical waves are emitted to the sample tray to uniformize the distribution of coffee particles therein. Subsequently, at least one frame of raw image is acquired for the uniformly distributed coffee particles.

The vibration frequency, vibration amplitude, or vibration duration of the vibration source may be fixed or adjustable. In some examples, the display interface of the device for analyzing coffee particles is further provided with adjustment options for vibration frequency, vibration amplitude, or vibration duration, enabling the user to select the corresponding vibration frequency, vibration amplitude, or vibration duration according to the size of the coffee particles. In some examples, the device for analyzing coffee particles can preliminarily determine the particle size of the coffee particles based on the analysis results of one or more previous frames of images collected by the camera, and automatically adjust the corresponding vibration amplitude or vibration duration accordingly.

In practical applications, the higher the temperature of coffee particles, the higher the energy of infrared radiation they emit outward. This, in turn, causes certain interference to image detection, affecting chromaticity measurement. In some examples, in step S702, one frame of initial representative chromaticity map is specifically obtained from the at least one frame of raw image. Additionally, a temperature sensor is provided in the coffee particle colorimetric analysis device; the current ambient temperature is acquired via the temperature sensor, and the chromaticity compensation method corresponding to the current ambient temperature is determined from chromaticity compensation methods applicable to different temperatures. The representative chromaticity map of the coffee particles is then determined based on the chromaticity values in the initial representative chromaticity map of the coffee particles and the chromaticity compensation method. Specifically, the current ambient temperature may refer to the sensor temperature, the light source temperature, or the temperature of the coffee particles.

The temperature sensor may be disposed close to the sample tray, enabling the measured temperature to be closer to that of the coffee particles in the sample tray. Chromaticity compensation methods for different temperatures can be obtained through pre-shipment calibration and stored in the device for analyzing coffee particles. Specifically, during calibration, the differences between the chromaticity values calculated at multiple ambient temperatures and those calculated at a reference temperature can be determined as chromaticity compensation values. The representative chromaticity map of the coffee particles is determined based on the initial representative chromaticity values in the initial representative chromaticity map of the coffee particles and the chromaticity compensation method. Specifically, each initial representative chromaticity value in the initial representative chromaticity map is adjusted by adding or subtracting the chromaticity compensation value corresponding to the current ambient temperature, with the resulting values serving as the representative chromaticity values to form the representative chromaticity map.

In the prior art, coffee particle colorimetric analysis devices generally use the chromaticity value of coffee particles when they reach temperature equilibrium as the calculation basis. When the temperature of the coffee particles is higher than that of the device, the temperature difference between the two will cause errors in chromaticity value measurement. In the embodiments of the present application, by pre-acquiring chromaticity compensation methods corresponding to multiple temperature points of the coffee particles, chromaticity compensation values under non-temperature equilibrium conditions are determined to perform temperature compensation.

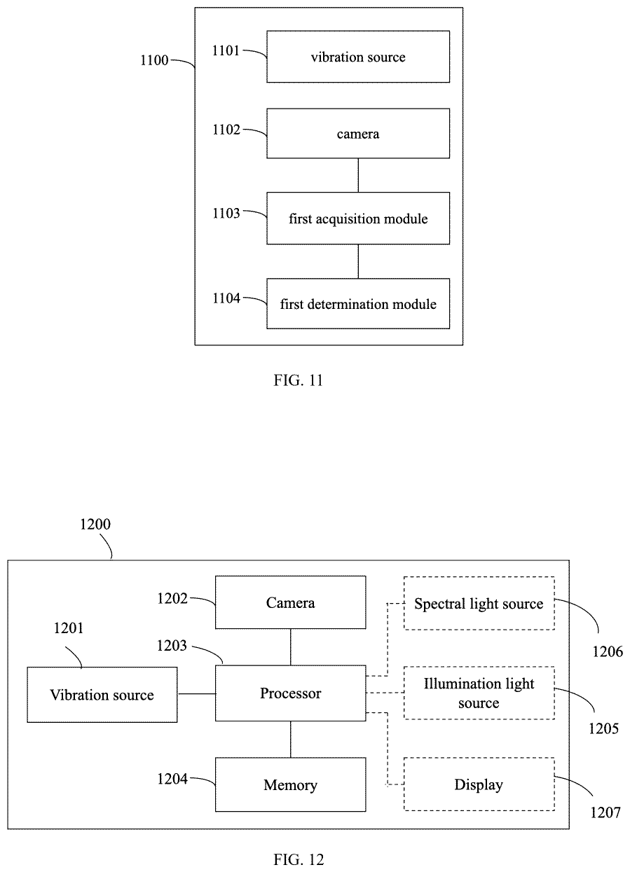

The present application also provides an apparatus for analyzing coffee particles, as shown in FIG. 11, which is an exemplary diagram of an embodiment of the device for analyzing coffee particles of the present application. The device for analyzing coffee particle 1100 includes a vibration source 1101, a camera 1102, a first acquisition module 1103, and first determination module 1104.

The vibration source 1101 is configured to drive the coffee particles to vibrate at least twice.

The camera 1102 is configured to respectively acquire images of the coffee particles after the at least two vibrations, obtaining a set of images to be detected having coffee particles with different distributions.

The first acquisition module 1103 is configured to respectively acquire initial recognition information of coffee particles in the images to be detected of the image set to be detected.

The first determination module 1104 is configured to determine final recognition information of the coffee particles based on the initial recognition information of coffee particles in at least some frames of the image set to be detected.

In some examples, the vibration source 1101 is configured to drive the coffee particles to vibrate in a first driving mode to obtain coffee particles with a first distribution; the camera 1102 is configured to capture images of the coffee particles with the first distribution, obtaining a first image to be detected; the vibration source 1101 is also configured to drive the coffee particles with the first distribution to vibrate in a second driving mode, obtaining coffee particles with a second distribution; the camera 1102 is also configured to capture images of the coffee particles with the second distribution, obtaining a second image to be detected.

In some examples, the first driving mode and the second driving mode are different; the control module 1101 is further used to control the vibration source to drive the coffee particles with the first distribution to vibrate in the second driving mode at least once after controlling the vibration source to drive the coffee particles with the first distribution to vibrate in the second driving mode; the camera 1102 is also used to capture images of the coffee particles after each driving in the second driving mode, to obtain at least one frame of image to be detected.

In some examples, at least one of the following items is different between the first driving mode and the second driving mode: vibration frequency, vibration amplitude, vibration duration, and vibration area.

In some examples, the vibration frequency in the first driving mode is a resonance frequency, and the vibration frequency in the second driving mode is less than or greater than the resonance frequency; and/or the vibration amplitude in the first driving mode is greater than the vibration amplitude in the second driving mode.

In some examples, the vibration amplitude in the first driving mode is greater than the vibration amplitude in the second driving mode, and the vibration duration in the second driving mode is longer than the vibration duration in the first driving mode.

In some examples, the second driving mode is determined according to the first image to be detected.

In some examples, the device 1100 further includes: a second determination module, used to determine at least one of the vibration frequency, vibration amplitude, vibration duration or vibration area in the second driving mode according to the initial recognition information of the coffee particles in the first image to be detected; the initial recognition information of the coffee particles in the first image to be detected includes the number and/or area of the coffee particles in the first image to be detected.

In some examples, the initial recognition information of the coffee particles in the image to be detected includes the number of coffee particles; the device 1100 further includes: a third determination module, used to determine the change in the number of the coffee particles based on the number of coffee particles in at least one frame of the image to be detected before the first image to be detected, the first image to be detected, and the second image to be detected; a fourth determination module, used to determine the driving mode of the vibration source after the second image to be detected based on the change in the number.

In some examples, determining the driving mode of the vibration source after the second image to be detected based on the quantity change includes at least one of the following: when the quantity change is a decrease in quantity or the change value is less than a threshold value, stopping the next drive of the vibration source on the coffee particles or continuing the next drive of the coffee particles with the second driving mode; or, when the quantity change is an increase in quantity and the change value is greater than the threshold value, continuing the next drive of the coffee particles with the first driving mode.

In some examples, the number of coffee particles in at least one frame of image to be detected before the first image to be detected, the first image to be detected, and the second image to be detected is the number of particles whose area is greater than a preset critical value in at least one frame of image to be detected before the first image to be detected, the first image to be detected, and the second image to be detected.

In some examples, the initial recognition information includes at least one of the particle size, quantity, area, volume, mass, and chromaticity of the coffee particles; and/or at least one of the quantity, area, volume, mass, and chromaticity of tiny coffee particles, wherein the tiny coffee particles are coffee particles having a particle size smaller than a first preset particle size, or a particle size smaller than the first preset particle size and greater than a preset critical value, wherein the value of the first preset particle size is smaller than the value of the particle size in the multiple particle size intervals.

In some examples, the initial recognition information further includes: at least one of the number distribution, area distribution, volume distribution, mass distribution, and chromaticity distribution of the coffee particles in different particle size ranges; and/or information on the proportion of the tiny coffee particles in all coffee particles.

In some examples, the final recognition information includes at least one of a final number distribution, a final area distribution, a final volume distribution, a final mass distribution, and a final chromaticity distribution of the coffee particles in different particle size intervals; the device 1100 further includes:

The display module is used to display at least one of the final number distribution, final area distribution, final volume distribution, final mass distribution, and final chromaticity distribution of the coffee particles in different particle size ranges on an interactive interface.

In some examples, the initial recognition information of the coffee particles includes the particle size of the coffee particles.

The device 1100 further includes: a second acquisition module, used to acquire a distortion function before determining the final recognition information of the coffee particles based on the initial recognition information of the coffee particles in at least some frames of the images to be detected in the image set to be detected, wherein the distortion function is used to indicate particle size compensation values at multiple pixel positions; a distortion correction module, used to perform distortion correction on the particle size of at least some of the coffee particles in the images to be detected in the image set to be detected according to the pixel positions of the coffee particles and the corresponding particle size compensation values to obtain the particle size of the coffee particles after distortion correction; the first determination module is specifically used to determine the final recognition information of the coffee particles based on the particle size of the coffee particles in at least some frames of the images to be detected in the image set to be detected after distortion correction.

In some examples, the initial recognition information of the coffee particles includes the particle size of the coffee particles; before determining the final recognition information of the coffee particles based on the initial recognition information of at least part of the frames of the image to be detected in the image set to be detected, the method further includes: a third acquisition module for acquiring a particle size compensation function, wherein the particle size compensation function is used to indicate the particle size compensation value under multiple brightness levels; a fourth acquisition module for respectively acquiring the particle size compensation values under multiple brightness levels; The first determining module is used to determine the final recognition information of the coffee particles according to the compensated particle size of the coffee particles in at least some of the frames of the images to be detected.

In some examples, the initial recognition information of the coffee particles includes the particle size of the coffee particles; the method further includes a calibration module for entering a calibration mode, wherein, in the calibration mode, the calibration module is further used to: capture an image of a calibration pattern with a preset area located at a preset position within the field of view to obtain a calibration image; obtain the number of pixels corresponding to the calibration pattern; determine a calibration size corresponding to one pixel based on the preset area and the number of pixels; the first acquisition module is specifically used to: respectively obtain the number of pixels of the coffee particles in the image to be detected in the image set to be detected; determine the particle size of the coffee particles based on the calibration size and the number of pixels.

In some examples, the image to be detected is an image captured when the coffee particles are illuminated by an illumination light source; the device further includes: a fifth acquisition module, used to acquire at least one frame of raw image, the at least one frame of raw image comprising raw pixel values of the image captured when the coffee particles are illuminated by at least one light source different from the illumination light source; a sixth acquisition module, used to acquire a frame of representative chromaticity diagram based on the at least one frame of raw image; and a fifth determination module, used to determine the overall chromaticity value of the coffee particles based on the one frame of representative chromaticity diagram.

The present application also provides a device 1200 for analyzing coffee particles, as shown in FIG. 12, which is another example diagram of an embodiment of the device for analyzing coffee particles of the present application. The device 1200 comprises: a vibration source 1201 configured to drive the coffee particles to vibrate at least twice; a camera 1202 configured to respectively capture images of the coffee particles after the at least two vibrations, obtaining a to-be-detected image set containing images of coffee particles with different distributions; one or more processors 1203 communicatively coupled to the camera 1202; and a memory 1204 storing instructions executable by the one or more processors 1203, wherein the instructions, when executed by the one or more processors 1203, cause the one or more processors 1203 to: acquire initial recognition information of the coffee particles in each to-be-detected image of the to-be-detected image set; and determine final recognition information of the coffee particles based on the initial recognition information of the coffee particles in at least some frames of to-be-detected images in the to-be-detected image set. In some examples, the camera 1202 comprises a photosensitive array.

In some examples, the vibration source is configured to drive the coffee particles to vibrate in a first driving mode, resulting in coffee particles with a first distribution, and to drive the coffee particles with the first distribution to vibrate in a second driving mode, resulting in coffee particles with a second distribution. The camera is configured to capture an image of the coffee particles with the first distribution, obtaining a first to-be-detected image, and to capture an image of the coffee particles with the second distribution, obtaining a second to-be-detected image; wherein the vibration frequency and/or vibration amplitude applied to the coffee particles in the first driving mode is higher than the vibration frequency and/or vibration amplitude applied to the coffee particles in the second driving mode.

In some examples the vibration source is configured to, after driving the coffee particles with the first distribution to vibrate in the second driving mode, further drive the coffee particles to vibrate in the second driving mode at least once more; and the camera is configured to further capture an image of the coffee particles respectively after each vibration driven in the second driving mode, obtaining at least one frame of to-be-detected image.

In some examples, at least one of vibration frequency, vibration amplitude, vibration duration, or vibration area in the second driving mode is determined based on the initial recognition information of the coffee particles in the first to-be-detected image, wherein the initial recognition information of the coffee particles in the first to-be-detected image includes the quantity and/or area of the coffee particles in the first to-be-detected image.

In some examples, the initial recognition information of the coffee particles in the to-be-detected image includes the quantity of the coffee particles. The one or more processors are further caused to determine a change in the quantity of the coffee particles based on the quantity of the coffee particles in at least one frame of to-be-detected image prior to the first to-be-detected image, the first to-be-detected image, and the second to-be-detected image. The vibration source is further configured to stop the next driving of the coffee particles or continue the next driving of the coffee particles in the second driving mode when the quantity change is a decrease in quantity or the change value is less than a threshold; or to continue the next driving of the coffee particles in the first driving mode when the quantity change is an increase in quantity and the change value is greater than the threshold.

In some examples, the initial recognition information includes at least one of the following: at least one of particle size, quantity, area, volume, mass, and chromaticity of the coffee particles; at least one of quantity distribution, area distribution, volume distribution, mass distribution, and chromaticity distribution of the coffee particles in different particle size intervals; at least one of quantity, area, volume, mass, and chromaticity of tiny coffee particles, where the tiny coffee particles are coffee particles with a particle size smaller than a first preset particle size, or with a particle size smaller than the first preset particle size and larger than a preset critical value;
information on the proportion of the tiny coffee particles among all coffee particles. The final recognition information includes at least one of final quantity distribution, final area distribution, final volume distribution, final mass distribution, and final chromaticity distribution of the coffee particles in different particle size intervals. The device 1200 further comprises a display 1207 configured to display at least one of the final quantity distribution, final area distribution, final volume distribution, final mass distribution, and final chromaticity distribution of the coffee particles in different particle size intervals.

In some examples, the initial recognition information of the coffee particles includes the particle size of the coffee particles. The one or more processors are further caused to: acquire a distortion function prior to determining the final recognition information of the coffee particles, where the distortion function indicates particle size compensation values at multiple pixel positions;
perform distortion correction on the particle size of at least some coffee particles in the to-be-detected images of the to-be-detected image set based on the pixel positions of the coffee particles and the corresponding particle size compensation values, so as to obtain the distortion-corrected particle size of the coffee particles. The final recognition information of the coffee particles is determined based on the distortion-corrected particle size of the coffee particles in at least some frames of to-be-detected images in the to-be-detected image set.

In some examples, the initial recognition information of the coffee particles includes the particle size of the coffee particles. The one or more processors are further caused to: acquire a particle size compensation function prior to determining the final recognition information of the coffee particles, where the particle size compensation function indicates particle size compensation values under multiple brightness levels; respectively acquire the brightness of the regions where the coffee particles are located in at least some frames of to-be-detected images in the to-be-detected image set; for the at least some frames of to-be-detected images, compensate the particle size of the coffee particles in the to-be-detected images based on the brightness of the regions where the coffee particles are located in the to-be-detected images and the particle size compensation values, so as to obtain the compensated particle size of the coffee particles. The final recognition information of the coffee particles is determined based on the compensated particle size of the coffee particles in at least some frames of to-be-detected images in the to-be-detected image set.

In some examples, the device is configured with a calibration mode, wherein in the calibration mode, the camera is configured to capture an image of a calibration pattern with a preset area and located at a preset position within the field of view to obtain a calibration image, and the one or more processors are further caused to acquire the number of pixels corresponding to the calibration pattern, and determine a calibration size corresponding to one pixel based on the preset area and the number of pixels. The initial recognition information of the coffee particles includes the particle size of the coffee particles determined based on the calibration size and the number of pixels of the coffee particles in each to-be-detected image of the to-be-detected image set.

In some examples, the device 1200 further comprises an illumination light source 1205 and at least one spectral light source 1206 different from the illumination light source, wherein the to-be-detected image is an image captured when the coffee particles are illuminated by the illumination light source.

The camera is further configured to capture at least one frame of raw image, which contains raw pixel values of images captured from the coffee particles when the coffee particles are respectively illuminated by the at least one spectral light source. The one or more processors are further caused to obtain one frame of representative chromaticity map based on the at least one frame of raw image, and to determine the overall chromaticity value of the coffee particles based on the one frame of representative chromaticity map.

In some examples, the one or more processors are further caused to: acquire at least one frame of initial raw image; determine invalid pixel values in the at least one frame of initial raw image, where the invalid pixel values refer to pixel values corresponding to objects other than the coffee particles in the initial raw image; remove the invalid pixel values from the at least one frame of initial raw image to obtain the at least one frame of raw image.

In some examples, the one or more processors are further caused to: determine positions of invalid pixel values based on the at least some frames of to-be-detected images, and determine the invalid pixel values in the at least one frame of initial raw image based on the positions of invalid pixel values.

In some examples, the at least one spectral light source includes n types of spectral light sources with different emission spectra, and the at least one frame of raw image includes n frames of raw images respectively corresponding to the n types of spectral light sources, where n is an integer greater than or equal to 2. The n types of spectral light sources include a first spectral light source and a second spectral light source, the emission spectrum of the first spectral light source includes a wavelength of 850 nm, and the dominant wavelength of the emission spectrum of the second spectral light source is a wavelength other than 850 nm. The one or more processors are further caused to generate n frames of chromaticity maps respectively corresponding to the n frames of raw images, acquire weights of the n types of spectral light sources, and generate the one frame of representative chromaticity map based on the weights of the n types of spectral light sources and the n frames of chromaticity maps, wherein the weight of the first spectral light source is higher than the weight of the second spectral light source.

In some examples, the one or more processors are further caused to: obtain one frame of initial representative chromaticity map based on the at least one frame of raw image; acquire the current ambient temperature; determine a chromaticity compensation value based on the current ambient temperature from chromaticity compensation values corresponding to different temperatures; and determine the representative chromaticity map of the coffee particles based on the initial representative chromaticity values in the initial representative chromaticity map of the coffee particles and the chromaticity compensation value.

In some examples, the one or more processors are further caused to: for a k-th type of spectral light source, acquire a basis function corresponding to the k-th type of spectral light source, where the basis function is a relational function between raw pixel values collected under illumination of the k-th type of spectral light source and corresponding chromaticity values, and k is any integer from 1 to n; acquire raw pixel values in a k-th frame of raw image, the k-th frame of raw image being the raw image collected when the coffee particles are illuminated by the k-th type of spectral light source; acquire corresponding chromaticity values based on the raw pixel values in the k-th frame of raw image and the basis function corresponding to the k-th type of spectral light source, so as to obtain the chromaticity map corresponding to the k-th frame of raw image.

In some examples, the one or more processors are further caused to: acquire at least one frame of initial raw image; acquire a brightness compensation function; determine a raw pixel compensation value for a pixel position based on the brightness compensation function and the pixel position in the initial raw image; compensate the raw pixel value at the pixel position in the at least one frame of initial raw image based on the raw pixel compensation value for the pixel position.

In some examples, the one or more processors are further caused to acquire the representative chromaticity distribution of the representative chromaticity map; the device further comprises a display configured to display the representative chromaticity distribution and the overall chromaticity value.

In some examples, the device further comprises a bearing surface for carrying the coffee particles, an illumination light source, and a backlight source. The vibration source is disposed on one side of the bearing surface; the illumination light source is disposed on the side of the bearing surface where the coffee particles are carried, and the backlight source is disposed on the other side of the bearing surface. The camera is configured to capture the to-be-detected image of the coffee particles on the bearing surface when the illumination light source and the backlight source emit light beams.

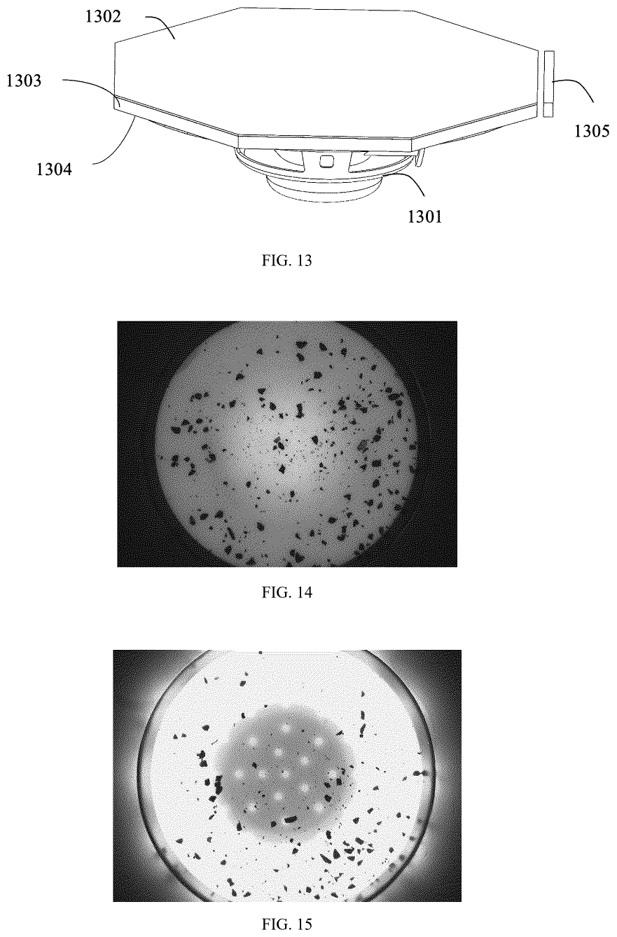

As shown in FIG. 13, FIG. 13 is a schematic diagram of the structure of the vibration source, the bearing surface, and the backlight source in one embodiment of the device for analyzing coffee particles of the present application. In some examples, the vibration source 1301 includes a power amplifier, or includes at least two linear vibration sources in different directions. The description of the vibration source can refer to the relevant description above. In FIGS. 13, the vibration source 1301 is illustrated by taking a power amplifier as an example.

In some examples, the bearing surface 1302 is a first light homogenizing film. The device further comprises a second light homogenizing film 1303 and a light guide plate 1304 disposed between the vibration source 1301 and the first light homogenizing film 1302, wherein the first light homogenizing film 1302, the light guide plate 1304, and the second light homogenizing film 1303 are arranged sequentially side by side, the light guide plate 1304 is located in a sealed space enclosed by the first light homogenizing film 1302 and the second light homogenizing film 1303, and the backlight source 1305 is disposed at the periphery of the light guide plate 1304.

In some examples, the second light-homogenizing film 1303 and the light guide plate 1304 are respectively connected to the first light-homogenizing film 1302 by glue to form a closed air cavity. The second light-homogenizing film 1303 is connected to the vibration source 1301. When the vibration of the power amplifier 1301 is transmitted to the second light-homogenizing film 1303, the vibration of the second light-homogenizing film 1303 is transmitted to the first light-homogenizing film 1302 through the air cavity, thereby driving the vibration of the coffee particles. In some examples, at least one through hole 1304 is also provided on the light guide plate 1304 to facilitate the transmission of vibration through the air cavity.

In particle size analysis, if only the illumination light source is turned on, since the illumination light source and the camera are located on the same side of the bearing surface, the light beam of the illumination light source acts as top light relative to the bearing surface. This results in a relatively dark background of the to-be-detected image, as shown in FIG. 14. FIG. 14 is a schematic diagram of the to-be-detected image captured by the camera when only the illumination light source is turned on. Due to the dark background of the to-be-detected image, errors may occur when distinguishing the background from the coffee particles, thereby reducing the accuracy of the coffee particle size analysis. By arranging a backlight source on the side of the bearing surface opposite to the photosensitive array, and turning on both the backlight source and the illumination light source simultaneously during particle size measurement, the problem of a dark background can be better solved. As shown in FIG. 15, FIG. 15 is a schematic diagram of the to-be-detected image captured by the camera when both the illumination light source and the backlight source are both turned on. It can be seen that the backlight source enables more accurate distinction between the background and the coffee particles, thus improving the accuracy of the coffee particle size analysis. Furthermore, to address the issue of uniform bottom backlighting, a light guide plate is also used in this example. The backlight source emits light from the side of the light guide plate, which can improve the brightness uniformity of the to-be-detected image and further enhance the accuracy of the coffee particle size analysis.

In some examples, at least two spectral light sources are included and configured to emit light with different wavelengths ranging from 500 nm to 1100 nm. And the camera is configured to capture frames of raw image when the at least two spectral light sources respectively irradiate the coffee particles.

In some examples, a preset calibration color card is further disposed inside the device for analyzing coffee particles, such as the preset calibration color card described in FIG. 10. For instance, the device further includes a hollow cylindrical structural member with an opening on one side, and a base detachably fixed to the opening of the structural member. The aforementioned light sources and camera are disposed on the inner top of the structural member, and the base includes a bearing surface and a vibration source. The preset calibration color card is fixed on the inner side surface of the structural member, located within the field of view of the camera, and does not block the coffee particles on the bearing surface.

The various embodiments of the present application have been described above, and the above description is exemplary, not exhaustive, and is not limited to the disclosed embodiments. Without departing from the scope and spirit of the described embodiments, many modifications and changes are obvious to those of ordinary skill in the art. The choice of terms used herein is intended to best explain the principles of the embodiments, practical applications, or improvements to the technology in the market, or to enable other persons of ordinary skill in the art to understand the embodiments disclosed herein.

What is claimed is:

1. A device for analyzing coffee particles, characterized by comprising:
a camera configured to respectively capture images of the coffee particles after the at least two vibrations, obtaining a to-be-detected image set containing images of coffee particles with different distributions;
a memory storing instructions executable by the one or more processors, wherein the instructions, when executed by the one or more processors, cause the one or more processors to:
acquire initial recognition information of the coffee particles in each to-be-detected image of the to-be-detected image set;

2. The device according to claim 1, wherein the vibration source is configured to drive the coffee particles to vibrate in a first driving mode, resulting in coffee particles with a first distribution, and to drive the coffee particles with the first distribution to vibrate in a second driving mode, resulting in coffee particles with a second distribution; and
the camera is configured to capture an image of the coffee particles with the first distribution, obtaining a first to-be-detected image, and to capture an image of the coffee particles with the second distribution, obtaining a second to-be-detected image;

3. The device according to claim 2, wherein the vibration source is configured to, after driving the coffee particles with the first distribution to vibrate in the second driving mode, further drive the coffee particles to vibrate in the second driving mode at least once more; and
the camera is configured to further capture an image of the coffee particles respectively after each vibration driven in the second driving mode, obtaining at least one frame of to-be-detected image.

4. The device according to claim 2, wherein at least one of vibration frequency, vibration amplitude, vibration duration, or vibration area in the second driving mode is determined based on the initial recognition information of the coffee particles in the first to-be-detected image, wherein the initial recognition information of the coffee particles in the first to-be-detected image includes the quantity and/or area of the coffee particles in the first to-be-detected image.

5. The device according to claim 4, wherein the initial recognition information of the coffee particles in the to-be-detected image includes the quantity of the coffee particles;
the to-be-detected image prior to the first to-be-detected image, the first to-be-detected image, and the second to-be-detected image;

6. The device according to claim 1, wherein the initial recognition information includes at least one of the following:
at least one of quantity distribution, area distribution, volume distribution, mass distribution, and chromaticity distribution of the coffee particles in different particle size intervals;
wherein the final recognition information includes at least one of final quantity distribution, final area distribution, final volume distribution, final mass distribution, and final chromaticity distribution of the coffee particles in different particle size intervals;
the device further comprises a display configured to display at least one of the final quantity distribution, final area distribution, final volume distribution, final mass distribution, and final chromaticity distribution of the coffee particles in different particle size intervals.

7. The device according to claim 1, wherein the initial recognition information of the coffee particles includes the particle size of the coffee particles;
acquire a distortion function prior to determining the final recognition information of the coffee particles, where the distortion function indicates particle size compensation values at multiple pixel positions;
wherein the final recognition information of the coffee particles is determined based on the distortion-corrected particle size of the coffee particles in at least some frames of to-be-detected images in the to-be-detected image set.

8. The device according to claim 1, wherein the initial recognition information of the coffee particles includes the particle size of the coffee particles;
the one or more processors are further caused to:
acquire a particle size compensation function prior to determining the final recognition information of the coffee particles, where the particle size compensation function indicates particle size compensation values under multiple brightness levels;
respectively acquire the brightness of the regions where the coffee particles are located in at least some frames of to-be-detected images in the to-be-detected image set;
for the at least some frames of to-be-detected images, compensate the particle size of the coffee particles in the to-be-detected images based on the brightness of the regions where the coffee particles are located in the to-be-detected images and the particle size compensation values, so as to obtain the compensated particle size of the coffee particles;
wherein the final recognition information of the coffee particles is determined based on the compensated particle size of the coffee particles in at least some frames of to-be-detected images in the to-be-detected image set.

9. The device according to claim 1, wherein the device is configured with a calibration mode, wherein in the calibration mode, the camera is configured to capture an image of a calibration pattern with a preset area and located at a preset position within the field of view to obtain a calibration image,
the one or more processors are further caused to acquire the number of pixels corresponding to the calibration pattern, and determine a calibration size corresponding to one pixel based on the preset area and the number of pixels; and
the initial recognition information of the coffee particles includes the particle size of the coffee particles determined based on the calibration size and the number of pixels of the coffee particles in each to-be-detected image of the to-be-detected image set.

10. The device according to claim 1, wherein the device further comprises an illumination light source and at least one spectral light source different from the illumination light source, wherein the to-be-detected image is an image captured when the coffee particles are illuminated by the illumination light source;
the camera is further configured to capture at least one frame of raw image, which contains raw pixel values of images captured from the coffee particles when the coffee particles are respectively illuminated by the at least one spectral light source;

11. The device according to claim 10, wherein the one or more processors are further caused to:
acquire at least one frame of initial raw image;
determine invalid pixel values in the at least one frame of initial raw image, where the invalid pixel values refer to pixel values corresponding to objects other than the coffee particles in the initial raw image;

12. The device according to claim 11, wherein the one or more processors are further caused to:
determine positions of invalid pixel values based on the at least some frames of to-be-detected images, and
determine the invalid pixel values in the at least one frame of initial raw image based on the positions of invalid pixel values.

13. The device according to claim 10, wherein the at least one spectral light source includes n types of spectral light sources with different emission spectra, and the at least one frame of raw image includes n frames of raw images respectively corresponding to the n types of spectral light sources, where n is an integer greater than or equal to 2;
the n types of spectral light sources include a first spectral light source and a second spectral light source, the emission spectrum of the first spectral light source includes a wavelength of 850 nm, and the dominant wavelength of the emission spectrum of the second spectral light source is a wavelength other than 850 nm;
the one or more processors are further caused to:
generate n frames of chromaticity maps respectively corresponding to the n frames of raw images, and
acquire weights of the n types of spectral light sources, and generate the one frame of representative chromaticity map based on the weights of the n types of spectral light sources and the n frames of chromaticity maps, wherein the weight of the first spectral light source is higher than the weight of the second spectral light source.

14. The device according to claim 10, wherein the one or more processors are further caused to:
obtain one frame of initial representative chromaticity map based on the at least one frame of raw image;
acquire the current ambient temperature;
determine a chromaticity compensation value based on the current ambient temperature from chromaticity compensation values corresponding to different temperatures; and

15. The device according to claim 10, wherein the one or more processors are further caused to:
for a k-th type of spectral light source, acquire a basis function corresponding to the k-th type of spectral light source, where the basis function is a relational function between raw pixel values collected under illumination of the k-th type of spectral light source and corresponding chromaticity values, and k is any integer from 1 to n;
th frame of raw image, the k-th frame of raw image being the raw image collected when the coffee particles are illuminated by the k-th type of spectral light source; and
th frame of raw image and the basis function corresponding to the k-th type of spectral light source, so as to obtain the chromaticity map corresponding to the k-th frame of raw image.

16. The device according to claim 10, wherein the one or more processors are further caused to:
acquire at least one frame of initial raw image;
acquire a brightness compensation function;
determine a raw pixel compensation value for a pixel position based on the brightness compensation function and the pixel position in the initial raw image; and
compensate the raw pixel value at the pixel position in the at least one frame of initial raw image based on the raw pixel compensation value for the pixel position.

17. The device according to claim 10, wherein the one or more processors are further caused to acquire the representative chromaticity distribution of the representative chromaticity map;
the device further comprises a display configured to display the representative chromaticity distribution and the overall chromaticity value.

18. The device according to claim 1, wherein the device further comprises a bearing surface for carrying the coffee particles, an illumination light source, and a backlight source;

19. The device according to claim 18, wherein the bearing surface is a first light homogenizing film;
the device further comprises a second light homogenizing film and a light guide plate disposed between the vibration source and the first light homogenizing film, wherein the first light homogenizing film, the light guide plate, and the second light homogenizing film are arranged sequentially side by side, the light guide plate is located in a sealed space enclosed by the first light homogenizing film and the second light homogenizing film, and the backlight source is disposed at the periphery of the light guide plate.

20. A method for analyzing coffee particles, characterized by comprising:
controlling a vibration source to drive the coffee particles to vibrate at least twice,
respectively capturing images of the coffee particles after the at least two vibrations, obtaining a to-be-detected image set containing coffee particles with different distributions;
